(19) United States
(12) Patent Application Publication
(10) Pub. No.: US 2025/0235262 A1
(43) Pub. Date: Jul. 24, 2025

(54) DEVICES, SYSTEMS, AND METHODS FOR REMOVING MATERIALS FROM A BODY

(71) Applicant: BOSTON SCIENTIFIC SCIMED, INC., MAPLE GROVE, MN (US)

(72) Inventors: Michael S H Chu, Brookline, MA (US); Gene Thomas Storbeck, Millis, MA (US); Kian S. Lim, Shrewsbury, MA (US)

(73) Assignee: BOSTON SCIENTIFIC SCIMED, INC., MAPLE GROVE, MN (US)

(21) Appl. No.: 19/035,444

(22) Filed: Jan. 23, 2025

Related U.S. Application Data
(60) Provisional application No. 63/624,405, filed on Jan. 24, 2024.

(51) Int. Cl. A61B 18/26; A61B 1/00; A61B 1/01; A61B 1/018; A61B 1/307; A61B 18/00

(52) U.S. Cl. CPC A61B 18/26; A61B 1/00119; A61B 1/01; A61B 1/018; A61B 1/307; A61B 2018/00184; A61B 2018/00714; A61B 2018/00982; A61B 2218/002; A61B 2218/007

(57) ABSTRACT

A fragmenting and/or fragment-removing system and associated methods configured to remove large fragments from an anatomical site not capable of being aspirated through standard lumens insertable to the anatomical site. In some aspects, the system includes a distal inlet channel, a proximal inlet channel, and an outlet channel. The relative positions of at least the distal inlet channel and the outlet channel may be varied, such as depending on the size of the materials to be removed by the system. The outlet channel may be defined within a lumen of the first sheath, between the inner surface of a first sheath and the outer surface of a tubular elongate member extending through the first sheath. A biasing member may be provided to maximize a size of the outlet channel by biasing the tubular elongate member to one side of the lumen of the first sheath.

### CROSS-REFERENCE TO RELATED APPLICATIONS

This application claims the benefit of priority under 35 U.S.C. § 119 to U.S. Provisional Application No. 63/624,405, filed Jan. 24, 2024, the entire disclosure of which is hereby incorporated by reference herein for all purposes.

### FIELD

The present disclosure relates generally to the field of medical devices, systems, and methods for removing materials, such as solid materials, from a patient's body. More particularly, the present disclosure relates generally to the field of medical devices, systems, and methods for removing solid materials such as a bodily mass and/or portions thereof, from a patient's body, such as from a body cavity. In some aspects, the present disclosure further relates to fragmenting and removing the bodily mass.

### BACKGROUND

Various medical conditions involve the presence of undesired bodily masses, such as hard stones, soft stones, impacted stones, tissue, tumors, etc., which are removable via surgical intervention, such as to remedy the condition. The bodily masses may be physically disrupted and reduced in size (to facilitate removal thereof) by a fragmenting device configured to direct energy, such as electric, hydraulic, laser, mechanical, ultrasound, etc., energy, at the bodily mass. For instance, various devices, such as lasers and/or laser fibers, can be inserted into an anatomical structure located (e.g., an anatomical cavity, such as a kidney) at which an undesired bodily mass (e.g., a kidney stone) is located to fragment and/or to dust the bodily mass to a smaller size capable of being more readily removed. For example, lithotripsy devices may be used to direct pulsed light energy at kidney stones. The energy is converted into mechanical and thermal energy, such as in the form of a cavitation bubble associated with the occurrence of a shockwave. The energy (e.g., cavitation bubble) may help to fragment, disrupt, cauterize, cut, break up, pulverize, etc., the kidney stone(s). In some aspects, the fragmenting device is inserted and advanced to the bodily mass through a lumen (e.g., working channel) of a tubular elongate member (e.g., a medical scope such as a ureteroscope). Devices can be inserted through the lumen of the tubular elongate member and advanced to the remnants of the bodily mass (e.g., smaller sized stones and/or stone fragments) to remove the remnants. For instance, retrieval devices, such as retrieval baskets, may be advanced to the remnants to remove the remnants. However, it may be difficult and time consuming to capture fine-sized fragments (e.g., smaller than 3 mm). Flushing the dust particles or using a popcorning technique (e.g., breaking down fragments of the bodily mass by impacting the fragments against one another or another structure) can scatter fragments/particles, particularly if located within an anatomical cavity, making it more difficult and time consuming to remove the unwanted material from the patient's body. Moreover, once the materials are captured by a retrieval device, the removal process, when feasible, may be time consuming as well. Typically, the retrieval device, along with the tubular elongate member, must be entirely withdrawn from the human body each time remnants of the bodily mass are captured by a retrieval device, particularly if the fragments are large to fit through the lumen of the tubular elongate member (e.g., working channel of the medical scope). The tubular elongate member and retrieval device are then reinserted, to capture and remove the next fragment. Repeated insertion and removal of the retrieval device and tubular elongate member becomes very time consuming and prolongs the procedure. Large bodily masses and/or fragments thereof may sometimes be too large to be readily removed and may clog removal devices. Although aspiration devices may be used to retrieve the remnants of the bodily mass, the bodily mass must be dusted to a very small size to be aspirated through the typically small diameter (e.g., relative to the initial size of the bodily mass) of the aspiration device lumen. Again, such process is time consuming. Typically, locating smaller stones for aspiration and/or stabilizing a small stone for lasing becomes more difficult as the bodily mass is fragmented. Solutions to these and other challenges in the art would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system is configured for removing fragmented remains of a bodily mass from an anatomical site within a patient. In some aspects, the system includes a first sheath defining a first lumen therethrough and having an inner surface and an outer surface; a tubular elongate member extending through the first lumen, the tubular elongate member defining a distal inlet channel therethrough and having an inner surface and an outer surface; and a biasing member within the first lumen maintaining spacing between the inner surface of the first sheath and the outer surface of the tubular elongate member. In some aspects, the first lumen is fluidly couplable with a suction source to define an outlet channel between the inner surface of the first sheath and the outer surface of the tubular elongate member; and the biasing member is insertable into the first lumen independently of the tubular elongate member.

In some aspects, the first sheath, the tubular elongate member, and the biasing member are movable with respect to one another to adjust the relative positions of distalmost ends thereof with respect to one another. In some aspects, the first sheath has a distalmost end advanceable distal to a distalmost end of the tubular elongate member to create a funnel within a distal end of the first sheath within the first lumen and between the distalmost end of the first sheath and the distalmost end of the tubular elongate member.

In some aspects, the biasing member includes a sensor.

In some aspects, a sensor is insertable into the first lumen independently of the tubular elongate member and the biasing member.

In some aspects, the tubular elongate member is a medical scope with a working channel defining the distal inlet channel.

In some aspects, the system further includes a second sheath defining a second lumen therethrough and having an inner surface and an outer surface, the first sheath positionable within the second lumen defined through the second sheath, wherein a proximal inlet channel is defined between the inner surface of the second sheath and the outer surface of the first sheath.

In some aspects, the system further includes a fragmenting device extendable through the tubular elongate member. In some aspects, the fragmenting device is movable with respect to the first sheath and the tubular elongate member between a position distal to a distalmost end of the first sheath and a position proximal to the distalmost end of the first sheath and within the first lumen.

In some aspects, the system further includes a fluid management system operatively associated with at least one of the first lumen or the distal inlet channel to control pressure and/or temperature conditions at the anatomical site.

In accordance with various principles of the present disclosure, a system is configured for removing fragments of a bodily mass from an anatomical site. In some aspects, the system includes an outer sheath defining an outer system lumen therethrough and having an inner surface and an outer surface; an inner sheath defining an inner system lumen therethrough and having an inner surface and an outer surface; and a tubular elongate member extending through the inner system lumen and defining a distal inlet channel therethrough fluidly couplable with a fluid source to irrigate the anatomical site. In some aspects, a space defined within the outer system lumen and between the inner surface of the outer sheath and the outer surface of the inner sheath defines a proximal inlet channel fluidly couplable with a second fluid source to irrigate the anatomical site; and a space defined within the inner system lumen and between the inner surface of the inner sheath and the outer surface of the tubular elongate member defines an outlet channel couplable with a suction source to aspirate a bodily mass or fragments thereof from the anatomical site.

In some aspects, the system further includes a fragmenting device extendable through the tubular elongate member.

In some aspects, the system further includes a dilator insertable within at least one of the outer sheath or the inner sheath.

In some aspects, the system further includes a sensor operatively associated with one of the inner sheath or the tubular elongate member, or insertable separate from the inner sheath and the tubular elongate member.

In some aspects, the system further includes a fluid management system operatively associated with at least one of the proximal inlet channel, the distal inlet channel, or the outlet channel to control pressure and/or temperature conditions at the anatomical site.

In accordance with various principles of the present disclosure, a method of removing materials from an anatomical site includes applying suction to the anatomical site via an outlet channel defined through a first sheath; irrigating the anatomical site via a distal inlet channel defined through a tubular elongate member extendable through the first sheath; and irrigating the anatomical site via a proximal inlet channel, defined through a second sheath through which the first sheath and the tubular elongate member are extendable through.

In some aspects, the method further includes shifting at least one of the first sheath, the tubular elongate member, or the second sheath with respect to the others of the first sheath, the tubular elongate member, and the second sheath.

In some aspects, the method further includes fragmenting the materials with a fragmenting device.

In some aspects, the method further includes withdrawing the tubular elongate member proximally within the first sheath while applying suction through the outlet channel to virtually basket materials out of the anatomical site.

In some aspects, the method further includes biasing the tubular elongate member against an inner surface of the first sheath to maximize a dimension of the outlet channel transverse to a direction of fluid flow. In accordance with various principles of the present disclosure, a method for removing materials from an anatomical site includes sizing the material to fit into a sheath using the distal end or edge of the sheath as an indicator.

In accordance with various principles of the present disclosure, a method for removing materials from an anatomical site includes suctioning the material into a lumen formed of a laser resistant material, suctioning the material against a fragmenting device, fragmenting the material with the fragmenting device, and suctioning out the fragmented material.

In some aspects, the fragmenting device is a laser fiber. In some aspects, the fragmenting device extends from a lumen defined through a tubular elongate member such as a medical scope.

In some aspects, the fragmenting device is operated to fragment the material while simultaneously suctioning out fragmented material.

In some aspects, the temperature within the lumen is elevated. In some aspects, the elevation in temperature is caused by the fragmenting device. In some aspects, stone retropulsion is reduced.

In some aspects, continuous suctioning will suction fragmented materials against the tip of the fragmenting device to be fragmented to even smaller sizes until the entire material is suctioned out of the body. In some aspects, the fragmenting device is a laser fiber, and the fragmented materials are suctioned against the tip of the laser fiber to be lased into smaller sizes. In some aspects, the fragmenting and suctioning is performed without the need for virtual basketing.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of a fragmenting and/or fragment-removing system formed in accordance with aspects of the present disclosure and positioned with respect to an example of an embodiment of an anatomical site.

FIG. 1A illustrates a detail view of detail area 1A-1A in FIG. 1.

FIG. 1B illustrates an end view along line 1B-1B in FIG. 1.

FIG. 1C is an end view along line 1C-1C in FIG. 1.

FIG. 1D is a cross-sectional view along line 1D-1D in FIG. 1.

FIG. 2 illustrates an elevational view of an alternate arrangement of elements of a fragmenting and/or fragment-removing system formed in accordance with aspects of the present disclosure.

FIG. 3 illustrates an elevational view of example of an embodiment of an arrangement of a dilator with a fragmenting and/or fragment-removing system formed in accordance with various principles of the present disclosure and positioned with respect to an example of an embodiment of an anatomical site.

FIG. 4A illustrates an elevational view of an example of an embodiment of an arrangement of a dilator with a fragmenting and/or fragment-removing system formed in accordance with various principles of the present disclosure.

FIG. 4B illustrates a perspective view of the example of an embodiment of FIG. 4A.

FIG. 4C illustrates a top view of the example of an embodiment of FIG. 4A.

FIG. 5 illustrates an elevational view of an example of an embodiment of an arrangement of an outer sheath with a dilator in accordance with various principles of the present disclosure.

FIG. 6 illustrates an elevational view of another example of an embodiment of an arrangement of an outer sheath with a dilator in accordance with various principles of the present disclosure.

FIG. 7 illustrates an elevational view of another example of an embodiment of an arrangement of an outer sheath with a dilator in accordance with various principles of the present disclosure.

FIG. 8 illustrates an elevational view of another example of an embodiment of an arrangement of an outer sheath with a dilator in accordance with various principles of the present disclosure.

FIG. 9 illustrates an elevational view of an example of an embodiment of a fitting usable with a fragmenting and/or fragment-removing system formed in accordance with various principles of the present disclosure.

FIG. 10A illustrates a side elevational view of a seal usable with a fragmenting and/or fragment-removing system formed in accordance with various principles of the present disclosure.

FIG. 10B illustrates an end view of the seal of FIG. 10A.

FIG. 11A illustrates a side elevational view of a seal usable with a fragmenting and/or fragment-removing system formed in accordance with various principles of the present disclosure.

FIG. 11B illustrates an end view of the seal of FIG. 11A.

FIG. 12 illustrates an elevational view of a fragmenting and/or fragment-removing system formed in accordance with aspects of the present disclosure and positioned with respect to an example of an embodiment of an anatomical site.

FIG. 13 illustrates an elevational view of a fragmenting and/or fragment-removing system formed in accordance with aspects of the present disclosure and positioned with respect to an example of an embodiment of an anatomical site.

FIG. 13A illustrates a use position of the example of an embodiment of a fragmenting and/or fragment-removing system illustrated in FIG. 13 within detail area 13B.

FIG. 13B illustrates the detail area 13B of FIG. 13.

FIG. 13C illustrates a use position of the example of an embodiment of a fragmenting and/or fragment-removing system illustrated in FIG. 13 within detail area 13B.

FIG. 13D illustrates a use position of the example of an embodiment of a fragmenting and/or fragment-removing system illustrated in FIG. 13 within detail area 13B.

FIG. 13E illustrates a use position of the example of an embodiment of a fragmenting and/or fragment-removing system illustrated in FIG. 13 within detail area 13B.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “passage” is not limited to a circular cross-section. As used herein, a “free end” or “distalmost end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, devices, systems, and methods are described herein for removing bodily masses and/or fragmented bodily masses from an anatomical site. It will be appreciated that reference may be made interchangeably herein to terms such as (including other grammatical forms of terms such as) fragments, particles, pieces, segments, remnants, portions, parts, dust, etc., of a larger element, such as a bodily mass, without intent to limit unless otherwise specified. The devices, system, and method described herein are optionally also configured to fragment undesired bodily masses to be removed in accordance with various principles of the present disclosure. Various removal devices and systems disclosed herein are adapted to manage different-sized fragments. The aspiration devices may be used in conjunction with a fragmenting device configured to fragment the bodily mass into particles which may be more readily removed by the aspiration device. The fragmenting device may break the bodily mass into smaller sized fragments and/or into even smaller sizes (approximately 1 mm in size/diameter) typically referenced in the art as dust. Dust particles that are smaller than 250 microns (μm) can be suspended in fluid (e.g., water), while fragments larger than 250 microns will quickly sink in fluid. Aspiration devices formed in accordance with various principles of the present disclosure may be adjustable in size to be capable of removing fragments of varying sizes. Alternatively or additionally, aspiration devices formed in accordance with various principles of the present disclosure may be configured to be used in cooperation with a fragmenting device, such as to facilitate operation of a fragmenting device. In some aspects, the fragmenting device/system includes outlet channels through which fragments of the bodily masses are suctioned or aspirated out of the patient, or at least away from the initial site of the bodily mass. In some aspects, the aspiration device/system may simply be formed as an aspiration or outlet channel.

In some aspects, aspiration devices formed in accordance with various principles of the present disclosure are configured to maintain the appropriate pressure/fluid volume and/or temperature within the anatomical structure from which the bodily mass is to be removed. For instance, if the bodily mass is being removed from an anatomical cavity (e.g., a kidney), suction applied to aspirate fragments of the bodily mass may reduce the pressure within the anatomical cavity. In accordance with various principles of the present disclosure, a fragmenting and/or fragment-removing system includes one or more inlet channels, such as irrigation channels, and an outlet channel (in some embodiments, one or more outlet channels). In some aspects, a distal inlet channel and a proximal inlet channel are provided. In some aspects, the position of the distal end of the outlet channel through which fragments are removed is adjustable/movable with respect to the distal inlet channel and/or the proximal inlet channel.

In accordance with various principles of the present disclosure, a fragmenting device capable of delivering sufficient energy to fragment a bodily mass, may be positioned at an anatomical site to fragment a bodily mass along with an aspiration device. As such, aspiration of fragments may be performed during use of the fragmenting device to aspirate fragments as they are being created by the fragmenting device. Moreover, the fragmenting device may be delivered through a medical scope such as a ureteroscope, with the outlet channel positioned to aspirate materials as the fragmenting device pulverizes the bodily mass. As such, the devices, systems, and methods of the present disclosure avoid the need to remove, reinsert, and reposition the medical scope and fragmenting device to aspirate materials from the treatment site, as may be required, even multiple times, by current devices, systems, and methods. And, because the aspiration device of the present disclosure is adjustable, as noted above, the aspiration device may be adjusted to accommodate removal of large fragments which previously were too large to fit through previous aspiration devices. Moreover, because the adjustability of the aspiration device allows for removal of fragments which were too large to be removed by previous aspiration devices, particles may be removed adjacent or in the general vicinity of the fragmenting device and need not be flushed to another location for removal (e.g., to the ureteropelvic junction (UPJ) in the case of lithotripsy in a kidney) as had been required by previous devices. As used herein, terms of proximity such “in the general vicinity of” or “adjacent” should be readily understandable by those of ordinary skill in the art as close enough to affect operation of a device or system. For instance, as described herein, an outlet channel in the general vicinity of a fragmenting device allows aspiration of materials quickly enough so as not interfere with fragmenting operations. For instance, aspiration channels adjacent a fragmenting device may clear the path between the fragmenting device and the bodily mass being fragmented thereby, as the fragmenting device is being operated, so that fragments created during such operation do not in turn interfere with further operation of the fragmenting device. Alternatively or additionally, aspiration channels adjacent a fragmenting device may clear the visual path of a visualization device (such as a camera of an endoscope with which the fragmenting device is delivered) to be able to substantially continuously view the fragmenting process without interference by the fragments, dust, etc., caused by such process because the proximity of the aspiration channel allows sufficiently rapid clearing of the vicinity of the fragmenting device and the visualization device.

As may be appreciated, because aspiration of materials from the bodily mass generally applies negative pressure (e.g., suction, a vacuum source, etc.) to the treatment site, if the bodily mass is in a body cavity, care must be taken so that the cavity does not collapse from the aspiration being performed. For instance, excessive suction (negative pressure aspiration) may cause injury such as collapse, bruising, and/or bleeding, and/or may obscure the field of view (e.g., of a visualization device used during use of the fragmenting device) if pressure within the anatomical cavity is not carefully monitored and balanced, such as by refilling the anatomical cavity concurrently with aspiration of materials therefrom. Alternatively or additionally, anatomical cavities, such as within a kidney, may be filled with a relatively low volume of fluid (e.g., to maintain desired distension of an organ such as the kidney) and may be inadvertently over-filled (over-pressurized) from excess fluid if sufficient care is not taken to balance pressure within the anatomical cavity. Moreover, a low volume of fluid and/or excess application of energy to pulverize the bodily mass (e.g., from lasing), can overheat the anatomical site, potentially burning surrounding tissue. Other complications may occur if the delicate pressure balance within an anatomical structure is not carefully maintained. For instance, in the case of removal of materials from a kidney with irrigation and aspiration, high renal pelvic pressure (e.g., intrarenal pressure, or IRP, such as due to high irrigation rate) may cause such complications as renal damage, fever, systemic inflammatory response syndrome (SIRS), sepsis, and other adverse conditions. The medical professional would have to wait until homeostasis within the anatomical cavity is restored before the aspiration and/or fragmentation can be resumed.

The principles of the present disclosure address the above concerns by providing a device and system with an outlet channel (e.g., for aspiration, suction, vacuum/negative pressure, etc.), a distal inlet channel (e.g., for irrigation, flushing of debris, etc.), and a proximal inlet channel (e.g., for irrigation). The distal inlet channel is generally positioned distal to the proximal inlet channel and thus closer to the distalmost end (e.g., the free, terminal end) of the system. More particularly, the proximal inlet channel may generally remain proximal to the fragmenting device and/or the aspirating device (e.g., the distal opening of the outlet channel) during use, whereas the distal inlet channel may remain distal to the proximal inlet channel (and optionally also distal to the outlet channel) during use. In some aspects, the fragmenting device is a laser device which includes a laser fiber which may be powered by a laser console to supply sufficient energy to a distal end thereof to fragment or pulverize a bodily mass. It will be appreciated that terms such as fragment, pulverize, break up, etc., a bodily mass may be used interchangeably herein without intent to limit unless specified. In some embodiments, the laser is turned off by a signal from a pressure/temperature sensor operatively associated with the system, such as when the fluid level in the kidney is low. The distal inlet channel may supply/refill fluid to flush away and/or to suspend fragments of materials pulverized by the laser device and/or to provide cooling fluids to reduce elevated temperatures which may be created by the laser device. In some aspects, elevated temperatures and/or dust may be aspirated by the outlet channel, such as to clear the view of a visualization devices (e.g., of a medical scope).

In some aspects, the fragmenting device is a passive device (e.g., not independently steerable, although optionally steerable by another device, such as a medical scope through which the fragmenting device extends) which may be delivered to an anatomical site within a first tubular elongate member such as within a first sheath. A steerable flexible elongate member may be operably associated with (e.g., inserted into) the first tubular elongate member to provide steerability to the fragmenting device. In some aspects, the fragmenting device is a laser fiber. Alternatively or additionally, the fragmenting device extends through a lumen through the steerable flexible elongate member, such as through a working channel of a medical scope. In some aspects, the fragmenting device may be utilized while still within (proximal to the distalmost end of) the first tubular elongate member. In some aspects, the fragmenting device is operated after being advanced distal to the distalmost end of the steerable flexible elongate member. In some aspects, the fragmenting device is advanceable distal to the distalmost end of the first tubular elongate member. In some aspects, both the fragmenting device and the steerable flexible elongate member are advanceable distal to the distalmost end of the first tubular elongate member, such as prior to operating/activating the fragmenting device.

In some aspects, a visualization device (steerable or passive) is used to visualize operation of the fragmenting device, and/or other procedures performed with the system of the present disclosure. In some aspects, the visualization device is positioned in the vicinity of the fragmenting device to be sufficiently close to provide informative visualization of the fragmenting operation being performed. In such instances, the distal inlet channel may remain adjacent such visualization device, such as to flush debris from the viewing path of the visualization device. For instance, in some embodiments, the fragmenting device is delivered through a lumen (e.g., working channel) of a tubular elongate member which also delivers a visualization element. The distal inlet channel may be formed through the tubular elongate member, and, in some cases, may be coextensive (i.e., through) the lumen through which the fragmenting device is delivered. As such, the distal inlet channel may remain closer than the proximal inlet channel to the fragmenting device as the fragmenting device is advanced into the anatomical site. In some embodiments, such as described above, the fragmenting device may be delivered through a working channel of a medical scope (e.g., endoscope, ureteroscope, flexible ureteroscope, laparoscope, etc.) having a visualization device such as a camera, laser optics, etc. Optionally, the distal inlet channel remains adjacent the visualization device, such as immediately adjacent, particularly if the working channel of a medical scope defines the distal inlet channel (and, thus, the opening to the distal inlet channel is adjacent the distal end of the medical scope) and a visualization element is at the distal end of the medical scope.

In accordance with various principles of the present disclosure, the above-described first tubular elongate member (through which may be delivered a fragmenting device of a system formed in accordance with various principles of the present disclosure) may be delivered through a second tubular elongate member. As such, the first tubular elongate member may alternately be referenced herein as an inner tubular elongate member or inner sheath, and the second tubular elongate member may alternately be referenced herein as an outer tubular elongate member or outer sheath. The outer sheath may surround the inner sheath, thereby protecting the anatomical passage (e.g., ureter walls) from the inner sheath (e.g., from impact or injury by the inner sheath) as the inner sheath is maneuvered/inserted/withdrawn with respect to the target site (e.g., inside the ureter and kidney). The space between the inner sheath and the outer sheath may define the proximal inlet channel in accordance with various principles of the present disclosure. It will be appreciated that the outer sheath need not extend to the distalmost end of the inner sheath. In embodiments in which, as described above, a distal inlet channel is defined through the inner sheath, such as through a tubular elongate member extending therethrough, an opening to such inlet channel remains adjacent to the distalmost end of the inner sheath, thereby defining a distal inlet channel. In some aspects, the distalmost end of the proximal inlet channel remains distal to the distalmost end of the outlet channel. In some aspects, the distal inlet channel, defined between the outer sheath and the inner sheath, remains proximally spaced from the distalmost end of the inner sheath and thus proximal to the distal inlet channel. In some aspects, the proximal inlet channel is proximal and external to the distalmost end of the outlet channel. In some aspects, the distal inlet channel is the working channel of a medical scope. In some aspects, the distal inlet channel (e.g., defined by the medical scope) can be extended distally beyond the distal end of the outlet channel and/or retracted proximally into the outlet channel. In some aspects, the distal end of the medical scope may be retracted further back than the proximal inlet channel (e.g., to perform virtual basketing of a bodily mass). The proximal inlet channel provides sufficient irrigation fluid at such location to ensure proper operation of the system of the present disclosure (e.g., performing and meeting operational expectations thereof, such as by preventing overheating, collapse of an anatomical cavity, flushing debris, etc., such as described above). For instance, in embodiments in which a working channel of a medical scope defines a distal inlet channel, the distal end of the outer sheath (and thus the inlet opening to the proximal inlet channel defined between the outer sheath and the inner sheath) may remain proximal to the distalmost end of the inner sheath to allow the proximal inlet channel to meet operational expectations thereof.

In view of the above, a system formed in accordance with various principles of the present disclosure provides a fragmenting/pulverizing device with a distal- or forward-facing aspiration channel with direct scope vision and two inlet channels. The proximal inlet channel can be used to replenish fluid at the treatment site without the velocity or fluid pressure of the inlet flow disrupting/scattering stone fragments. The distal inlet channel (e.g., defined through a scope working channel) may also be used to replenish fluid at the treatment site. Alternatively or additionally, the distal inlet channel may be used to locally flush the vicinity of a visualization device to remove air bubbles, clear the field of view, move/relocate stones and/or debris, and/or to clear the lumen within the inner sheath. The medical professional may elect when to use each inlet channel-either individually/separately, or simultaneously.

In some aspects, principles of the present disclosure may be applied to treat (lase, pulverize, fragment, dust, remove, destroy, reduce the size of, etc.) renal stones. Renal stones can be of different sizes, shapes, hardnesses, etc., and can be located anywhere in the kidney or in the renal system. Various configurations of devices and systems described herein, including various combinations and subcombinations of elements or components thereof, may be used in accordance with various principles of the present disclosure, such as, for example, to treat renal stones and/or other bodily masses. Not all embodiments will be needed or used for a given treatment protocol. It will be appreciated that fragmenting (e.g., lasing) of renal stones or other bodily masses may be performed within the inner sheath (e.g., when positioned distal to the outer sheath), while larger masses generally would need to be fragmented outside the inner sheath and into smaller particles before they can be aspirated into the inner sheath. For example, bodily masses which are too large to fit within the outlet channel may be suctioned against the distal end of the inner sheath and lased or otherwise fragmented such as in that position. In some aspects, smaller bodily masses may be suctioned into the inner sheath and fragmented therein. In some aspects, the distal end or edge of the inner sheath is used as an indicator in sizing the bodily mass which may fit into the inner sheath. In some aspects, the inner sheath may be formed of a laser-resistant type material along a distal portion or optionally along the entire length thereof.

It will be appreciated that a medical professional may use other/additional equipment, tools, devices, etc., to facilitate stone fragmentation and/or for other tasks. In some aspects, various tools may be inserted through the working channel of a medical scope and/or may be used as a biasing member. Examples of additional tools which may be used include a retrieval basket or other tools to reposition a bodily mass. For instance, it may be desirable or medically indicated to move a renal stone from a lower pole to an upper pole of a kidney for easier access thereto. Alternatively or additionally, the medical professional may aspirate and/or irrigate during the fragmenting procedure (e.g., lasing) to clear the field of view and/or to suction out fragments and/or elevated temperatures concurrently and/or between fragmenting episodes and/or after fragmenting has been performed to aspirate out fragments of the bodily mass.

Various embodiments of devices, apparatuses, assemblies, systems, and methods for removing fragmented bodily masses and optionally also fragmenting a bodily mass will now be described with reference to examples illustrated in the accompanying drawings. The illustrated devices, apparatuses, assemblies, and/or systems include various elements, members, components, etc., configured to fragment and/or remove fragments of bodily masses from an anatomical site. It will further be appreciated that terms such as fragment, pulverize, break, lase, dust, crush, etc., including other grammatical forms thereof, may be used interchangeably herein without intent to limit (unless specifically indicated) to refer to reducing the size of a bodily mass. For the sake of convenience, and without intent to limit, the various systems described herein are referenced as fragmenting and/or fragment-removing systems. However, the various devices, apparatuses, assemblies, systems, etc., of the present disclosure may be configured to either fragment, or remove, or both fragment and remove bodily masses or parts thereof; and/or aspirate other materials; and/or irrigate treatment sites; and/or sense or otherwise determine conditions at a treatment site; and/or perform various other operations related to fragmenting and/or removing fragments from a treatment site. It will be appreciated that reference may be made to terms such as irrigate, flush, etc. (including other grammatical forms thereof), interchangeably and without intent to limit. It will further be appreciated that reference may be made to terms such as aspirate, suction, remove, apply vacuum or vacuum or negative pressure, etc. (including other grammatical forms thereof), interchangeably and without intent to limit.

In the accompany drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 1000 are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments.

It will be appreciated that reference herein to “one embodiment,” “an embodiment,” “some embodiments”, “other embodiments”, etc., indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

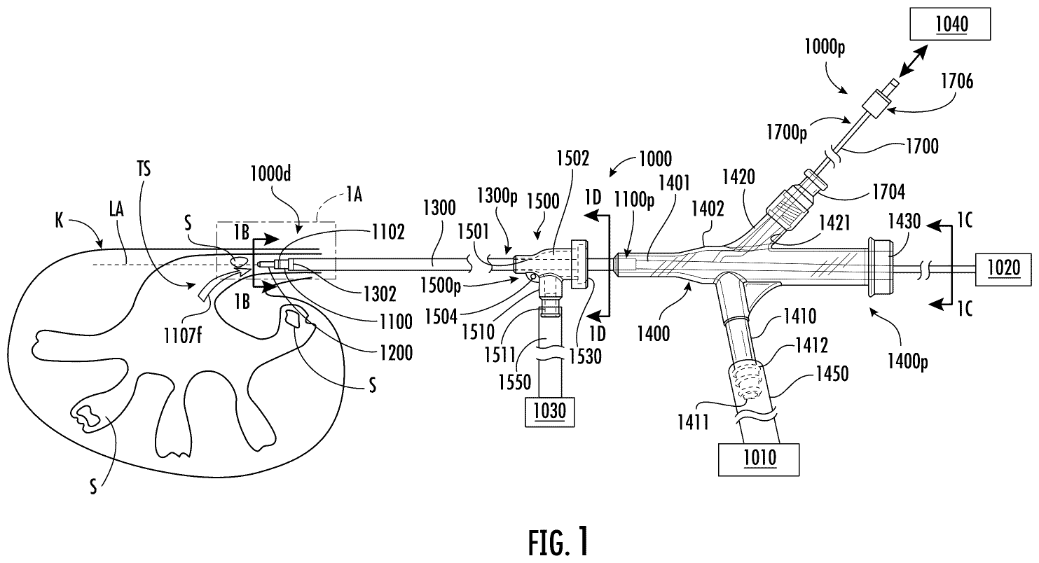

Turning now to the drawings, an example of an embodiment of a fragmenting and/or fragment-removing system 1000 formed in accordance with various principles of the present disclosure is illustrated in FIG. 1 with respect to a schematic example of an embodiment of a treatment site T. It will be appreciated that reference may be made herein to a treatment site, anatomical site, target site, etc., interchangeably and without intent to limit. Furthermore, it will be appreciated that reference made herein to an anatomical cavity as an example of an anatomical site, but the principles of the present disclosure need not be so limited. Although the example of an embodiment of a treatment site is illustrated herein as a kidney, and the system is thus used to eliminate kidney stones, it will be appreciated that devices, systems, assemblies, methods, etc., disclosed herein have other applications and utility as well.

The fragmenting and/or fragment-removing system 1000 illustrated in FIG. 1 includes a first sheath 1100 defining an outlet channel 1107 (see, e.g., the end view of FIG. 1B, showing the outlet channel 1107 into which suction flow 1107f, shown in FIG. 1, is directed) of the fragmenting and/or fragment-removing system 1000 through which a bodily mass and/or fragments thereof may be removed from a treatment site. The fragmenting and/or fragment-removing system 1000 optionally includes a tubular elongate member 1200 extending within/through the first sheath 1100. Additionally or alternatively, the fragmenting and/or fragment-removing system 1000 includes a second sheath 1300 extending over first sheath 1100 (i.e., through which the first sheath 1100 extends). The distal end 1000d of the fragmenting and/or fragment-removing system 1000 (including distal ends of one or more of the inner sheath 1100, the tubular elongate member 1200, and/or the outer sheath 1300) is insertable into a patient's body (e.g., transluminally, such as through a naturally-occurring body orifice; percutaneously; or otherwise) to a treatment site T. A body 1400 (e.g., main body, connector, handle, etc.) may be provided along a generally proximal end 1000p of the fragmenting and/or fragment-removing system 1000 to couple (e.g., to provide fluid connections among) various components of the fragmenting and/or fragment-removing system 1000, as described in further detail below. Optionally, the proximal end 1100p of the inner sheath 1100 is operatively coupled with the body 1400. Optionally, the proximal end 1300p of the outer sheath 1300 is operatively coupled with a fitting 1500, which, in turn, may be operatively coupled with the inner sheath 1100, as described in further detail below.

As illustrated in further detail in FIG. 1A, showing detail area 1A in FIG. 1, the first sheath 1100 defines a lumen 1101 therethrough. The lumen 1101 may be fluidly coupled at or along a proximal end 1100p of the first sheath 1100 with a suction source 1010, as illustrated schematically in FIG. 1. For instance, the proximal end 1100p of the first sheath 1100 may be fluidly coupled with a lumen 1401 defined through the body 1400/housing 1402 of the body 1400, and, in turn, with a lumen 1411 defined through a first sidearm 1410 (e.g., a straight sidearm) of the body 1400. In some aspects, the first sheath 1100 may be coupled with the housing 1400. In some aspects, the sidearm 1410 and the body 1400 are sealed to the atmosphere, even when additional instruments (e.g., a biasing member and/or a medical scope) are inserted therein. In some aspects, the lumen 1411 of the first sidearm 1410 extends transverse to the lumen 1101 defined through the inner sheath 1100. The lumen 1411 of the first sidearm 1410, in turn, may be fluidly coupled with a suction connector tube 1450 (e.g., engaged with barbs 1412 on the first sidearm 1410). The suction connector tube 1450 may be coupled, in turn, with the suction source 1010. The suction source 1010 may have any form or configuration such as known to those of ordinary skill in the art, and thus is schematically illustrated, the present disclosure not being limited in this regard. It will be appreciated that terms such as suction, aspiration, outlet, vacuum, vacuum pressure, removal, evacuation, etc., including various grammatical forms thereof, may all be used to refer to the withdrawal of the fragments, particles, dust, etc., of the bodily mass to be removed from the anatomical site. As fluidly coupled with a suction source 1010, the lumen 1101 of the first sheath 1100 may be considered to define an outlet channel 1107 of the fragmenting and/or fragment-removing system 1000.

The inner sheath 1100 and/or the outer sheath 1300 and/or the housing 1400 and associated fittings may be formed of a transparent or clear material, such as to allow viewing of materials aspirated therethrough, or an opaque material, the present disclosure not being limited in this regard. In some aspects, the inner sheath 1100 may have more than one section. For instance, in some embodiments, the inner sheath 1100 is made of at least of a distal section and a proximal section. The material of the distal section may be selected to be laser resistant, such as to withstand damage from a fragmenting device such as a laser 1600 which may be extended into and used within the inner sheath 1100 (as described in further detail below). For example, the distal section may be made of Expanded Polytetrafluoroethylene (EPTFE) or lined with EPTFE. Additionally or alternatively, the material of the distal section of the inner sheath 1100 may be soft and/or flexible, have high temperature stability (e.g., a melting point of at least about 380° C.), and/or made in different densities/column strengths. Optionally, the material of the distal section of the inner sheath 1100 may be reinforced, such as with an EPTFE coil. A radiopaque marker 1102 may be provided along the distal end 1100d of the inner sheath 1100, such as to facilitate visualization thereof (e.g., with fluoroscopy), such as during navigation, placement, use, etc., as described in further detail below. The proximal section of the inner sheath 1100 is operatively associated with the body 1400, thereby coupling the lumen 1101 defined through the inner sheath 1100 with the first sidearm 1410 of the body 1400, as described above. The lumen 1101 of the inner sheath 1100 may also be fluidly coupled with a second sidearm 1420 of the body 1400 which may be used for insertion of further devices through the lumen 1101, such as described in further detail below.

As illustrated in the detail view in FIG. 1A of the detail area 1A in FIG. 1, the optional tubular elongate member 1200 may extend (e.g., axially, longitudinally, etc.) through the lumen 1101 of the first sheath 1100. With such arrangement, the above-described outlet channel 1107 is defined between the inner surface 1103 of the first sheath 1100 (e.g., defining the inner diameter of the first sheath 1100) and the outer surface 1205 of the tubular elongate member 1200 (e.g., defining the outer diameter of the tubular elongate member 1200). The tubular elongate member 1200 may be inserted into the lumen 1101 of the inner sheath 1100 via the proximal end 1400p of the body 1400. More particularly, a seal 1430 may be operatively associated with the proximal end 1400p of the body 1400 and may include at least one through hole 1431 through which the tubular elongate member 1200 is insertable into the lumen 1101 of the inner sheath 1100. The through hole 1431 (see, e.g., the end view along line 1C-1C of FIG. 1, as illustrated in FIG. 1C) may be sized, shaped, configured, and/or dimensioned to sealingly engage the outer surface 1205 of the tubular elongate member 1200 to inhibit/prevent leakage. For example, the through hole 1431 may have a diameter slightly smaller than the outer diameter of the tubular elongate member 1200 such that when the tubular elongate member 1200 is inserted into the through hole 1431, the seal 1430 seals the outlet channel 1107 formed between the inner sheath 1100 and the tubular elongate member 1200. In some aspects, the seal 1400 is assembled, glued, tethered, or otherwise coupled with respect to the body 1400 such that it will not be lost, or needs be assembled (or misassembled) by the user. The seal may be screw threaded, friction fitted, or otherwise removably mounted on the proximal end 1400p of the body 1400, or fixedly mounted thereon (e.g., welded), the present disclosure not being limited in this regard. In some embodiments the seal is a Tuohy Borst adaptor

As illustrated in FIG. 1A, and in the end view of FIG. 1 illustrated in FIG. 1B, the tubular elongate member 1200 defines a lumen 1201 therethrough. The lumen 1201 may be fluidly coupled with a fluid source 1020, such as at a proximal end thereof. Although the proximal end of the tubular elongate member 1200 is not illustrated, the configuration thereof may be readily understood by to those of ordinary skill in the art, and thus does not require illustration to facilitate understanding thereof. The fluid source 1020 provides fluid (e.g., saline, etc.) for irrigation, flushing, etc., of the treatment site, such as known to those of ordinary skill in the art, and thus is schematically illustrated, the present disclosure not being limited in this regard. As fluidly coupled with a fluid source 1020, the lumen 1201 defines a first inlet channel 1207 of the fragmenting and/or fragment-removing system 1000. In some aspects, the distalmost end 1200t of the tubular elongate member 1200 is translatable between a proximal position, within the first sheath 1100 and proximal to the distalmost end 1100t of the first sheath 1100, and a distal position extending outside the first sheath 1100 and distal to the distalmost end 1100t of the first sheath 1100, such as to modify the position at which the first inlet 1207 supplies fluid into the treatment site T, as discussed in further detail below. In some aspects, the tubular elongate member 1200 may define a lumen defining a fluid inlet coupled to a fluid source that is separate from the lumen through which the fragmenting device 1600 extends.

In the illustrated example of an embodiment, the tubular elongate member 1200 is a medical scope. As may be appreciated by those of ordinary skill in the art, medical scopes have various components providing functionalities as known in the art for transluminal delivery to a treatment site within a patient's body. Examples of medical scopes include, without limitation, flexible ureteroscopes, arthroscopes, bronchoscopes, colonoscopes, cystoscopes, duodenoscopes, gastroscopes, hysteroscopes, laparoscopes, and ureteroscopes. In view of the example of an embodiment of a treatment site being a kidney, the illustrated example of an embodiment may be a flexible ureteroscope which may include a steering system (such as with pull wires manipulable to deflect the distal end of the scope shaft as needed during a procedure). However, a fragmenting and/or fragment-removing system 1000 formed in accordance with various principles of the present disclosure may include and/or use other types of medical scopes, the present disclosure not being limited in this regard. In the example of an embodiment illustrated in FIG. 1, FIG. 1A, and FIG. 1B, the lumen 1201 defining the first inlet channel 1207 of the tubular elongate member 1200 of the fragmenting and/or fragment-removing system 1000 is a working channel 1203 of a medical scope such as a ureteroscope. Moreover, in such example of an embodiment, the outer surface 1205 of the tubular elongate member 1200 is the outer surface 1203 of an insertion member of the medical scope.

As further illustrated in the detail view in FIG. 1A, the optional outer sheath 1300 defines a lumen 1301 defined therethrough and through which the inner sheath 1100 extends. In accordance with various principles of the present disclosure, the outer sheath 1300 may be fluidly coupled with a fluid source 1030. For instance, the proximal end 1300p of the outer sheath 1300 may be fluidly coupled with a fitting 1500, and, in turn, with a lumen 1511 port defined through a sidearm 1510 of the fitting 1500. The sidearm 1510, in turn, may be fluidly coupled with a suction source 1030, such as via a connection tube 1550. Fluid from the fluid source 1030 may flow through the space between the inner sheath 1100 (e.g., the outer surface 1105 of the inner sheath 1100, such as defining an outer diameter of the inner sheath 1100) and the outer sheath 1300 (e.g., the inner surface 1303 of the outer sheath 1300, such as defining the inner diameter of the outer sheath 1300), such space thereby defining a second inlet channel 1307 of the fragmenting and/or fragment-removing system 1000. Fluid from the fluid source 1030 can flow from the second inlet channel 1307 into the treatment site T (as an inlet of fluid) distally from the distalmost end 1300t of the outer sheath 1300 (via the space between the outer surface 1105 of the inner sheath 1100 and the inner surface 1303 of the outer sheath 1300 just proximal to the distalmost end 1300t) and/or via distal holes/apertures 1309 formed along a distal end 1300d of the outer sheath 1300 just proximal to the distalmost end 1300t of the outer sheath 1300. The fluid source 1030 may be the same as or different from the fluid source 1020 to which the tubular elongate member 1200 is fluidly coupled. In some aspects, fluid supply to the first inlet channel 1207 and to the second inlet channel 1307 may be separately and independently controllable, Preferably, if the tubular elongate member 1200 and the outer sheath 1300 are coupled with same fluid source, fluid supply to each may be separately and independently controlled so that the fluid flow through the first inlet channel 1207 (defined through the tubular elongate member 1200) may be different from the fluid flow through the second inlet channel 1307 (defined between the outer sheath 1300 and the inner sheath 1100), the benefits of such arrangement being appreciated with reference to the further descriptions thereof provided below. Because the second inlet channel 1307 is defined between the outer sheath 1300 and the inner sheath 1100 (and thus proximal to the distalmost end 1100t of the inner sheath 1100), the second inlet channel 1307 generally remains proximal to the first inlet channel 1207. Accordingly, the second inlet channel 1307 may be considered and thus alternately referenced herein as a proximal inlet channel 1307, and the first inlet channel 1207 may be considered and thus alternately referenced herein as a distal inlet channel 1207. It will be appreciated that in some embodiments, instead of defining an inlet channel, the outer sheath 1300 may define a drain channel between the inner surface 1303 thereof and the outer surface 1105 of the inner sheath 1100. In some aspects, the drain is not fluidly coupled with negative pressure and may be considered a passive drain. Furthermore, it will be appreciated that in some embodiments, an outer sheath 1300 is not used, and a system formed in accordance with various principles of the present disclosure may simply have a single inlet and a single outlet.

The outer sheath 1300 may be formed from a kink-resistant material. For instance, the distal end 1300d of the outer sheath 1300 may be more flexible than the proximal end 1300p of the outer sheath 1300, such as to facilitate insertion into the renal pelvis and/or to facilitate navigation thereof within a patient's body, such as to facilitate navigation of the inner sheath 1100 and/or the tubular elongate member 1200 which extend therethrough. In some aspects, at least a portion of both the distal end 1300d and the proximal end 1300p of the outer sheath 1300 may be reinforced, such as with a coil and/or braid. A radiopaque marker 1302 (such as illustrated in FIG. 1 and FIG. 1A) may be provided along the distal end 1300d of the outer sheath 1300, such as to facilitate visualization thereof (e.g., with fluoroscopy), such as during navigation, placement, use, etc., and/or to view the position of the distal end 1300d of the outer sheath 1300 with respect to the distal end 1100d of the inner sheath 1100. In some aspects, the outer sheath 1300 has a hydrophilic and/or friction-reducing coating over the outer surface 1305 thereof. A hydrophilic coating and/or friction-reducing coating on the outer sheath 1300 may facilitate insertion and advancement of the outer sheath 1300 into the patient. The use of a hydrophilic and/or friction-reducing coating may minimize discomfort and/or otherwise improve patient outcomes. These specialized sheaths offer numerous advantages over traditional sheaths, including improved patient comfort, reduced risk of infection, and faster recovery times.

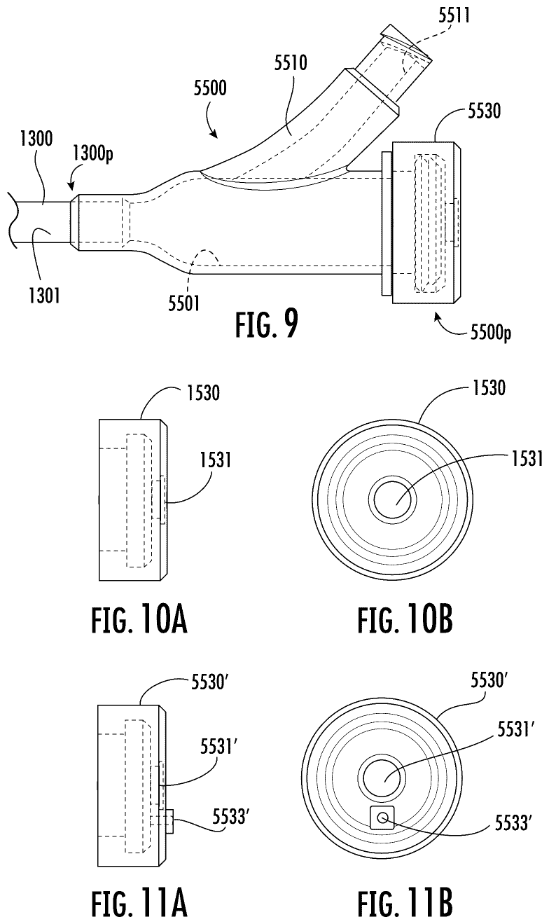

As described above, the proximal end 1300p of the outer sheath 1300 may be connected to a sidearm 1510 of a fitting 1500. The lumen 1511 defined through the sidearm 1510 may extend transverse to the lumen 1301 through the outer sheath 1300 and the lumen 1501 through the housing 1502 of the fitting 1500. The sidearm 1510 may extend such that the lumen 1511 therethrough is substantially perpendicular to the lumen 1501 through the fitting housing 1502 (such as in the example of an embodiment illustrated in FIG. 1), or may be at angle greater or less than 90° with respect to the outer sheath 1300 (such as in the example of an embodiment illustrated in FIG. 9 and described in further detail below). In some embodiments, a seal 1530 is provided at a proximal end 1500p of the fitting 1500 and fitted over the outer surface 1105 of the inner sheath 1100. The seal 1530 may have a through hole 1531 (such as illustrated in FIG. 1D) that is slightly smaller than the outer diameter of the outer surface 1105 of the inner sheath 1100, such as to seal the proximal inlet channel 1307 defined between the outer sheath 1300 and the inner sheath 1100. In some aspects, the seal 1530 may be attached to the outer sheath 1300, such as to prevent loss and/or to save an assembly step for the user. In some aspects, the outer sheath 1300 is advanceable further into the renal pelvis. Coupling of the seal 1530 to the housing 1502 may allow the advanceability of the outer sheath 1300. In some aspects, a suture hole 1504 is defined in the housing 1502 of the fitting 1500 configured for securing of a suture to the outer sheath 1300 and to the patient in a manner known those of ordinary skill in the art.

In accordance with various principles of the present disclosure, the inlet flow through the distal inlet channel 1207 counteracts the suction/aspiration flow through the outlet channel 1107, such as when the distalmost end 1200t of the tubular elongate member 1200 is retracted into the inner sheath 1200 and proximal to the distalmost end 1100t of the inner sheath 1100. One manner of avoiding or at least minimizing such counteraction is to lower or turn off the irrigation supplied by the distal inlet channel 1207, thereby allowing the suction through the outlet channel 1107 to dominate. In such instance, the replenishing fluid (fluid maintaining a desired homeostatic pressure at the treatment site T) delivered into the treatment site T is mainly delivered by the proximal inlet channel 1307.

In accordance with various principles of the present disclosure, as described above, and as may be appreciated, such as with reference to FIG. 1A and FIG. 1B, the outlet channel 1107 of the fragmenting and/or fragment-removing system 1000 illustrated in FIG. 1 (through which fragments are removed from a treatment site T) may be considered to be defined by (e.g., by the space between) the inner diameter of the inner sheath 1100 and the outer diameter of the tubular elongate member 1200. Further in accordance with various principles of the present disclosure, it may be desirable to maximize the size/area (e.g., generally in a direction transverse to the direction of flow through the outlet channel 1107, such as transverse to a longitudinal axis LA of the fragmenting and/or fragment-removing system 1000) of the outlet channel 1107. In order to maintain a desired spacing between the inner surface 1103 of the inner sheath 1100 and the outer surface 1205 of the tubular elongate member 1200, a biasing member 1700 may be positioned within the inner sheath 1100, such as illustrated in FIG. 1A and FIG. 1B, to maintain a space between the inner surface 1103 of the inner sheath 1100 and the outer surface 1205 of the tubular elongate member 1200. For instance, as illustrated in FIG. 1B, the biasing member 1700 may occupy one side of the lumen 1101 defined within the inner sheath 1100, while the tubular elongate member 1200 occupies the other side of the lumen 1101. In another words, the space between the inner surface 1103 of the inner sheath 1100 and the outer surface 1205 of the tubular elongate member 1200 (which may be referenced herein as a gap therebetween) is shifted or maximized by the presence of the biasing member 1700, positioning the tubular elongate member 1200 on the opposite side of the lumen 1101 than the location of the biasing member 1700 to allow larger fragments to pass out the outlet channel 1107 defined along the length of the inner sheath 1100 alongside the length of the biasing member 1700. In some aspects, the biasing member 1700 biases the tubular elongate member 1200 against a first area along the inner surface of the inner sheath 1100 to maximize a distance between a second area along the inner surface of the inner sheath 1100 opposite the first area to maximize a dimension of the outlet channel 1107 transverse to direction of fluid flow along the longitudinal axis LA of the fragmenting and/or fragment-removing system 1000. In some aspects, as illustrated in FIG. 1B, the biasing member 1700 has a generally circular profile, however, other shapes or configurations are within the scope of the present disclosure. In some aspects, one or more biasing members are inserted into the inner sheath 1100.

In some aspects, the biasing member 1700 is an elongate member, such as a flexible elongate member. In some aspects, the biasing member 1700 is a monofilament extrusion with a flexible distal end or a guidewire with a flexible distal end or the like. In some aspects, the biasing member is a microcatheter, a catheter, a balloon catheter, a retrieval device, an ultraviolet light (e.g., black light/UV-A light), a biopsy device, and/or or a medical elongated device. In some aspects, the biasing member 1700 has a flexible distal end 1700d. In some aspects, the biasing member 1700 may be a steerable elongate member. In some aspect the steerable elongate member is configurated with a visualization device (e.g., imager, such as a camera chip). In some aspects, the inner sheath 1100 is a passive element (i.e., not capable of actively being steered), and a steerable biasing member 1700 thus allows steering or other control and/or navigation of the inner sheath 1100 or at least the distal end 1100d thereof. The distal end 1700d of the biasing member 1700 may flex independently of the inner sheath 1100 (even if such flexion then causes the inner sheath 1100 to flex as well), as the biasing member 1700 need not be attached to the inner sheath 1100. In some aspects, the biasing member 1700 is formed of or at least covered with EPTFE (and/or another laser-resistant material, such as for safety).

In some aspects, a connector 1704, such as a Tuohy Borst adaptor, can be releasable coupled to the second sidearm 1420 of the body 1400 and/or the biasing member 1700 (e.g., to the exterior thereof), such as illustrated in FIG. 1, to allow adjustment of the length of the biasing member 1700 relative to the length of the inner sheath 1100. For instance, in the example of an embodiment illustrated in FIG. 1, the biasing member 1700 may be inserted into the fragmenting and/or fragment-removing system 1000, such as into the inner sheath 1100 thereof, with the assistance of the body 1400. More particularly, the biasing member 1700 may be inserted into a sidearm of the body 1400, such as the second sidearm 1420 (e.g., a curved sidearm 1420). The second sidearm 1420 has a lumen 1421 defined therethrough in fluid communication with the lumen 1401 defined through the body 1400/housing 1402 of the body 1400 and thus in fluid communication with the lumen 1101 defined through the inner sheath 1100. Insertion of the biasing member 1700 into the second sidearm 1420 of the body 1400 thus facilitates insertion of the biasing member 1700 into the inner sheath 1100 which extends into the body 1400, as described above. In some aspects, the biasing member 1700 may be extended distal to the distalmost end 1100t of the inner sheath 1100. However, when aspirating larger masses (e.g., masses closer to or even larger than the largest dimension of the outlet channel 1107), the biasing member 1700, and optionally also the tubular elongate member 1200, can be withdrawn slightly into the inner sheath 1100 (proximal to the distalmost end 1100t of the inner sheath 1100) to allow the entire outlet 1107 and/or the entire lumen 1101 of the inner sheath 1100 to accommodate a larger mass therein, such as described in further detail below.

In some aspects, the biasing member 1700 incorporates a sensor element 1702 capable of sensing and/or indicating one or more conditions at the treatment site T. For instance, the biasing member 1700 may include a temperature and/or pressure sensor 1702, such as at or along a distal end 1700d thereof. Alternatively or additionally, the sensor element 1702 includes a force sensor, an optical pressure sensor, a magnetic sensor, a color senor, a light sensor, a distance sensor, a time-of-flight sensor, and/or a biological-material sensing material (e.g., capable of sensing antibodies, enzymes, cell receptors which interacts with analyte(s), etc.). Other types of sensors may determine the size and composition of the bodily mass (e.g., renal stone) being targeted. The sensor element 1702 may include an optical, physicochemical, and/or piezoelectric transducer that translates the biological signal to electrical and/or electric signals. In some aspects, the biasing member 1700 with a sensor element 1702 may be mounted on (e.g., glued to) the outer surface of the tubular elongate member 1200. In some aspects, a sensor such as described above may be mounted on (e.g., glued to) the outer surface of the fragment-removing system 1000.

In the example of an embodiment illustrated in FIG. 1A and FIG. 1B, the biasing member 1700 is illustrated as a temperature and pressure (T/P) sensor element 1702. In some aspects, the sensor element 1702 is mounted inside a tube within the biasing member 1700 and exposed through a side window at the distal end 1700d of the biasing member 1700. For instance, the sensor element 1702 may be mounted inside a distal closed end tube such as an EPTFE tube with a side window. The outer diameter of the tube may bias the tubular elongate member 1200 off to one side of the lumen 1101 within the inner sheath 1100. The biasing member 1700 may be used with a tubular elongate member 1200 in the form of a medical scope with or without its own sensors (e.g., temperature and/or pressure sensors). A biasing member 1700 having a sensor element 1702 may be covered with an EPTFE material, coating, tube, etc., such that it is laser resistant (in water) for in-sheath lasing.

A biasing member 1700 with a sensor element 1702 can be operatively coupled with a fluid management system 1040 (FMS), with microcontrollers, regulators, solenoid valves, pumps, etc., such as known those of ordinary skill in the art (and thus illustrated schematically), to “automatically” regulate/control various aspects of the fragment-removing system 1000 and/or the treatment site T (such as the temperature and/or pressure of the treatment site T). For instance, when the fluid/pressure level within the kidney is low, a pressure sensor can send a signal to the laser of the system 1000 to pause delivery of energy to the laser so that the laser is not used in a “dry environment.” A sensor 1700 can provide, e.g., laser power settings and/or other information to the user in order to treat the patient safely and efficaciously. In some aspects, a connector 1706, such as an electrical plug, may be attached to the proximal end 1700p of the sensor 1700 to allow the sensor 1700 to be operatively coupled with fluid manage system 1040 (e.g., a box/console thereof). The fluid manage system 1040 may control the magnitude of fluid volume exchange between the treatment site T and any or all of the suction source 1010, first fluid source 1020, and/or second fluid source 1030. As may be appreciated, the fragmenting and/or fragment-removing system 1000 may include any of a variety of displays, such as monitors, displaying and/or otherwise providing the temperature and/or pressure at the treatment site T. In some aspects, various devices, systems, etc., such as electro-mechanical motors, may be used to pump fluid into and out of the fragmenting and/or fragment-removing system 1000 (e.g., in association with the suction source 1010, and/or the fluid sources 1020, 1030). Optionally, the medical professional may adjust the fluid volume exchange manually to regulate conditions such as, without limitation, pressure and temperature, at the treatment site T (e.g., within the kidney).

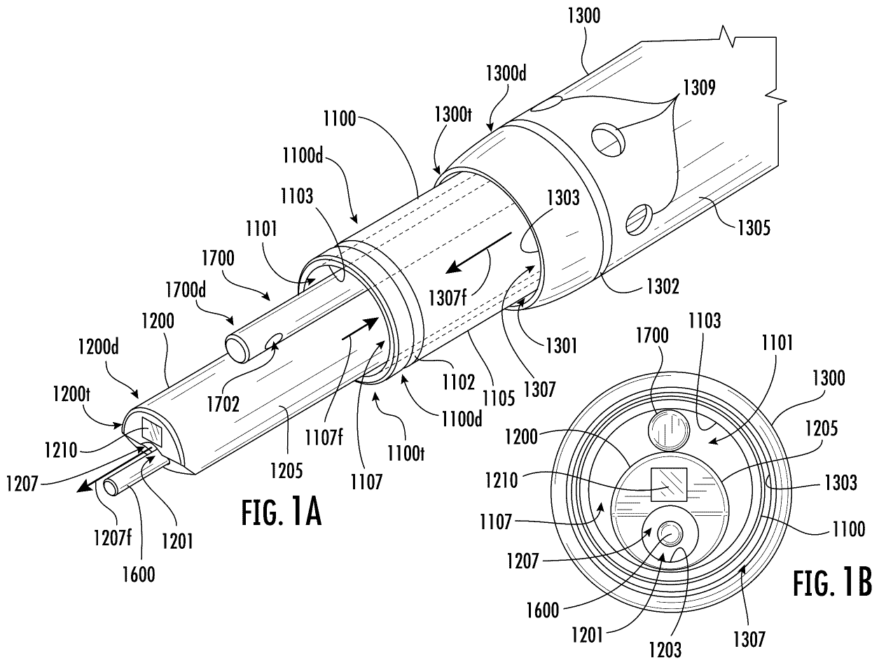

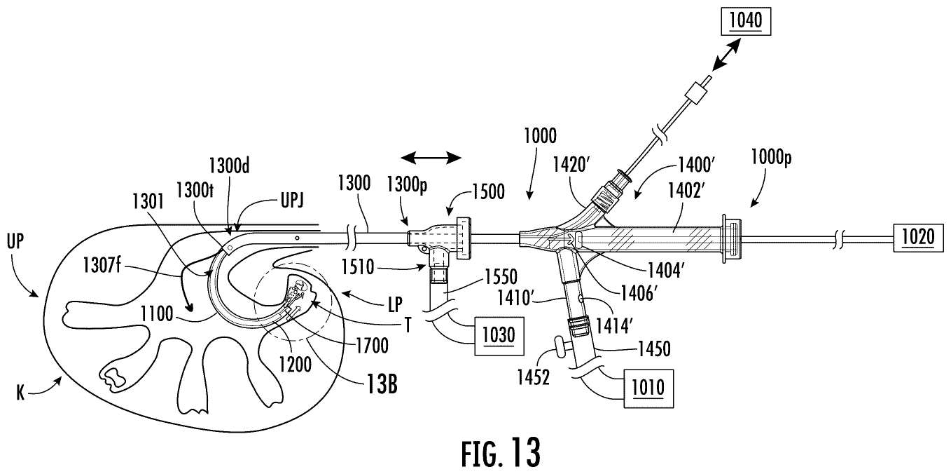

In embodiments with a biasing member 1700 which does not include a sensor, a separate sensor may be provided with the fragmenting and/or fragment-removing system 1000. For instance, as illustrated in FIG. 2, an example of an embodiment of a fragmenting and/or fragment-removing system 1000 (which may be substantially the same as the fragmenting and/or fragment-removing system 1000 illustrated in FIG. 1 other than the biasing member 1700) includes a separate sensor 1710 in addition to a biasing member 1700. As illustrated in FIG. 2, the separate sensor 1710 may be inserted into the fragmenting and/or fragment-removing system 1000 via the fitting 1500 (in contrast with entering the lumen 1101 of the inner sheath 1100 via the body 1400). More particularly, in the example of an embodiment of a fitting 1500 illustrated in FIG. 2, the fitting may include a seal 1530 similar to the seal 1430 described above. The seal 1530 may have a through hole for the separate sensor 1710 separate from (e.g., spaced from) the above-described through hole for the inner sheath 1100 (such as described below with reference to the example of an embodiment of a seal 5530 illustrated in FIG. 11A and FIG. 11B). The separate sensor 1710 may thus extend into the outer sheath 1300, such as alongside the inner sheath 1100. The separate sensor 1710 may be operatively coupled with a fluid manage system 1040 in a manner similar to that described above with respect to the biasing member 1700 with a sensor element 1702. Moreover, a separate sensor 1710 may otherwise be used and function similar to a sensor element 1702 provided with (e.g., carried or formed on) a biasing member 1700 as described above.

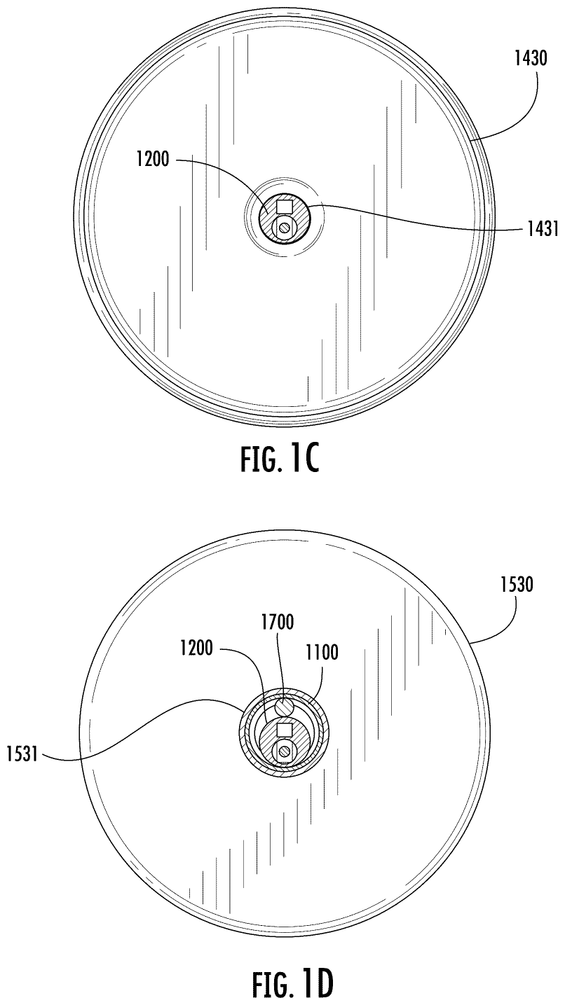

In some aspects, a dilator may be used to facilitate navigation of the distal end 1000d of a fragmenting and/or fragment-removing system 1000 formed in accordance with various principles of the present disclosure, and entry of the distal end 1000d with respect to the treatment site T. For instance, a dilator may be particularly useful in navigating through a generally fragile ureter and/or urethra and into a renal pelvis. In some aspects, a dilator may facilitate insertion of the fragment-removing system 1000 into a patient. In some aspects, at least the distal end (and optionally a portion of the or the full longitudinal extent) of the dilator may be covered with a hydrophilic and/or friction-reducing coating to facilitate insertion. In addition to, or instead of, facilitating access of a biasing member 1700 to a fragmenting and/or fragment-removing system 1000 formed in accordance with various principles of the present disclosure, the second sidearm 1420 of the body 1400 of the fragmenting and/or fragment-removing system 1000 may be used to facilitate insertion of a dilator with respect to the fragmenting and/or fragment-removing system 1000. For instance, as illustrated in FIG. 3, a dilator 1800, with an outer diameter just slightly smaller than the inner diameter of the inner sheath 1100, is inserted into the lumen 1101 of the inner sheath 1100 via the second sidearm 1420 of the body 1400. In some aspects, the dilator 1800 has a tapered distal end 1800d to facilitate entry thereof into a treatment site T (e.g., into the ureteral orifice in the example of an embodiment of a treatment site T as illustrated in FIG. 3). More particularly, in the example of an embodiment illustrated in FIG. 3, the distal end 1800d of the dilator 1800 has a tapered section 1802 tapering the outer diameter of the dilator 1800 from a first diameter to a second smaller second diameter along a distal section 1804 of the dilator 1800. As illustrated in FIG. 3, the distal section 1804d of the dilator 1800 is extended to the distal end 1000d of the fragmenting and/or fragment-removing system 1000 and distal to the distal end 1300d of the outer sheath 1300.

In some aspects, the dilator 1800 includes a dilator hub 1810 which can be releasably engaged (e.g., screw tightened, etc.) with respect to the second sidearm 1420 of the body 1400. The dilator 1800 may have a through lumen 1801 configured to accept a guidewire 1850 over which the dilator 1800 may be guided to the treatment site T. The guidewire 1850 may be directed to the treatment site T in a manner known those of ordinary skill in the art. In some aspects, the guidewire 1850 is inserted first, up into the kidney. The dilator 1800 may then be inserted through the second sidearm 1420 and locked. The tip of the dilator 1800 may be inserted onto the proximal end of already-placed guidewire 1850. The dilator system is advanced over the guidewire 1850 to the UPJ. The dilator 1800 and guidewire 1850 at this point may be removed. The biasing member 1700 may be inserted into the second sidearm 1420 and into the inner sheath 1100 and the tubular elongate member 1200 may be inserted through the proximal seal of the inner sheath 1100 towards the distal end of the inner sheath 1100. The tubular elongate member 1200 may be further advanced into the renal pelvis for examination. The sheath system can then be advanced over the deflected the tubular elongate member 1200 when needed.

In some aspects, the dilator 1800 is inserted into the inner sheath 1100, and the inner sheath 1100, with the dilator 1800 inserted therein, are inserted into the outer sheath 1300. For instance, in some aspects, the outer sheath 1300 is separate from the inner sheath 1100 and the outer sheath 1300 may be placed before the inner sheath 1100 is placed, such as with the distal end 1300d of the outer sheath 1300 already positioned at the treatment site T. The outer sheath 1300 in such case can protect the ureters as the inner sheath 1100 is maneuvered, such as to different calyxes of the kidney. In some aspects, a dilator 1800 such as illustrated in FIG. 3 is preassembled/preinserted into the inner sheath 1100 (e.g., by the manufacturer) so that the medical professional need not actively insert the dilator 1800 into the inner sheath 1100. In some embodiments, the outer sheath 1300 is a thin wall sheath and may be attached (e.g., at a tip thereof) to the inner sheath 1100 to prevent buckling of the outer sheath 1300 during delivery through the ureter and/or to prevent kinking of the (thin wall) outer sheath 1300 when the dilator 1800 is removed from the outer sheath 1300. In some embodiments, holes are provided at the distal end of the outer 1300 sheath for fluid to exit the proximal inlet channel 1307 when the outer sheath 1300 is attached to the inner sheath 1100. As may be appreciated, the dilator 1800 may be guided over the already or initially-placed guidewire 1850 to facilitate insertion of the fragmenting and/or fragment-removing system 1000 into the body and to the treatment site T (e.g., at least up to the UPJ in the illustrated example of an embodiment of FIG. 3). Once the distal end 1000d of the fragmenting and/or fragment-removing system 1000 has been positioned at the treatment site T, with the assistance of the dilator 1800 and the guidewire 1850, the dilator 1800 and the guidewire 1850 may be removed from the fragmenting and/or fragment-removing system 1000 and the patient. The tubular elongate member 1200 and the biasing member 1700 may then be inserted into the inner sheath 1100. For instance, in the example of an embodiment illustrated in FIG. 1, the biasing member 1700 is insertable into the inner sheath 1100 via the second sidearm 1420 of the body 1400, after the dilator 1800 has been withdrawn therefrom.

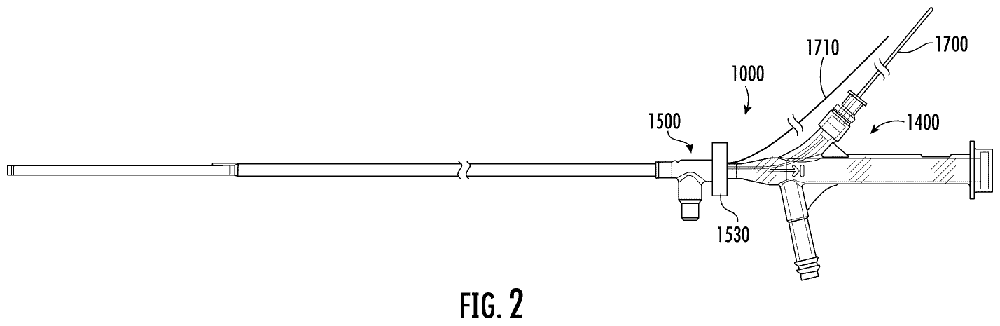

Instead of being inserted into the inner sheath 1100 via the second sidearm 1420 of the body 1400, such as illustrated in FIG. 3, a dilator 2800 may be inserted into the lumen 1101 of the inner sheath 1100 via the proximal end 1400p of the body 1400, such as illustrated in FIG. 4A, FIG. 4B, and FIG. 4C. More particularly, the example of an embodiment of a dilator 2800 illustrated in FIG. 4A, FIG. 4B, and FIG. 4C is inserted into the proximal end 1400p of the body 1400 which opens into the axial lumen 1401 of the body 1400 and into the lumen 1101 of the inner sheath 1100. In some aspects, the dilator 2800 may be coupled with the body 1400, such as to be maintained in place with respect to the inner sheath 1100. For instance, in the example of an embodiment illustrated in FIG. 4A, FIG. 4B, and FIG. 4C, the dilator 2800 may have a hub 2810 with one or more distal extensions 2812 configured to engage a hub or seal 1430 at the proximal end 1400p of the body 1400. In some aspects, the distal extensions 2812 include hooks 2814 configured to engage and to hook onto corresponding tabs 1434 on the hub/seal 1430 to maintain the dilator 2800 in place with respect to the body 1400.

As noted above, in some aspects, the inner sheath 1100 with a dilator 1800, 2800 already extending therein is inserted through the lumen 1301 of the outer sheath 1300. In some aspects, the inner sheath 1100 and dilator 1800, 2800 are inserted into the outer sheath 1300 after the distal end 1300d of the outer sheath 1300 has been inserted to a treatment site. The dilator 1800, 2800 may then be removed and a tubular elongate member 1200 and optional biasing member 1700 and/or optional independent sensor 1710 may be inserted into the inner sheath 1100 (e.g., the dilator 1800, 2800 may be exchanged with a tubular elongate member 1200 and/or a biasing member 1700 and/or optional independent sensor 1710).

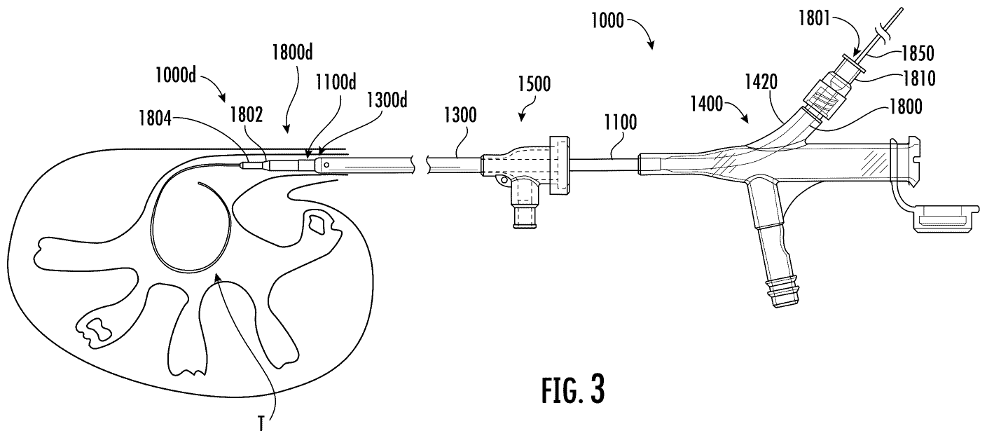

Alternatively, in some embodiments, a dilator is not inserted into the patient via the inner sheath 1100. Instead, a dilator may be inserted into the patient via the outer sheath 1300. In some aspects, a dilator may be inserted into the lumen 1301 of an outer sheath 1300 and optionally also coupled with respect to the outer sheath 1300 and/or a fitting operatively associated with the proximal end 1300p of the outer sheath 1300. For instance, in the example of an embodiment illustrated in FIG. 5, a dilator 1800 is inserted into the lumen 1301 of the outer sheath 1300 of a fragment-removing system and is releasably coupled to the outer sheath 1300 by means of a Tuohy Borst adaptor 1830. The Tuohy Borst adaptor 1830 optionally is coupled to the proximal end 1500p of a fitting 1500 operatively coupled with a proximal end 1300p of the outer sheath 1300. In some aspects, the Tuohy Borst adaptor 1830 is tightened with respect to a grommet 1840 having a through hole through which the outer sheath 1300 extends. In some aspects, the grommet 1840 is formed of an elastic material which is compressible to grip the outer surface 1805 of the dilator 1800, such as upon tightening the Tuohy Borst adaptor 1830. Loosening the Tuohy Borst adaptor 1830 may allow the grommet 1840 to expand and disengage from the outer surface 1805 of the dilator 1800. The Tuohy Borst adaptor 1830 may also be tightened or loosened to be an adjustable valve/seal which may accommodate a range of outer diameters of inner sheaths 1100 which may be passed therethrough and into the lumen 1300 of the outer sheath 1300. In some aspects, the outer sheath 1300 can be made to be held in the palm of one hand and operated with the thumb of the same hand. For instance, the Tuohy Borst adaptor 1830 can be operated with a single hand to be loosened or tightened (e.g., with respect to the outer sheath 1300).

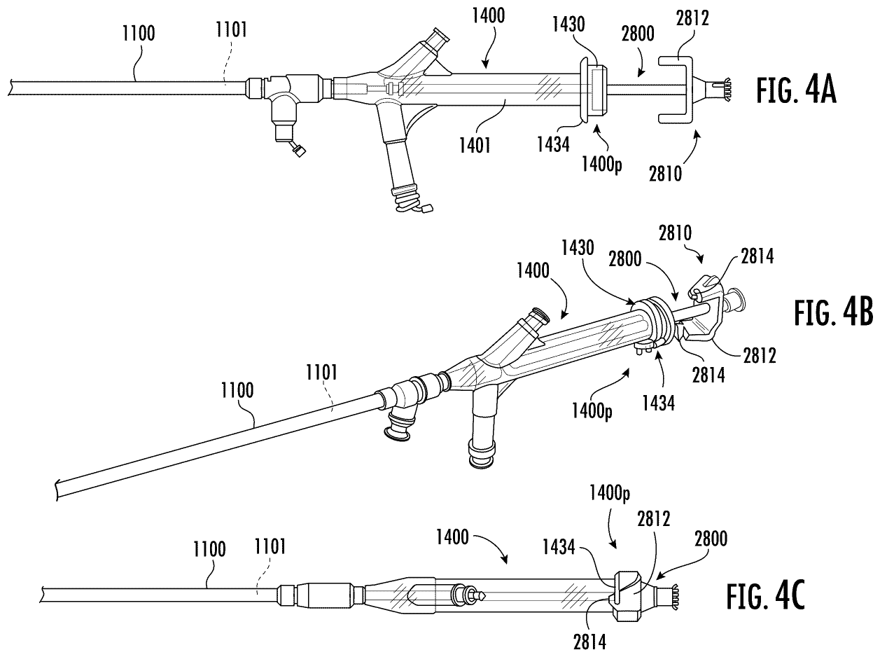

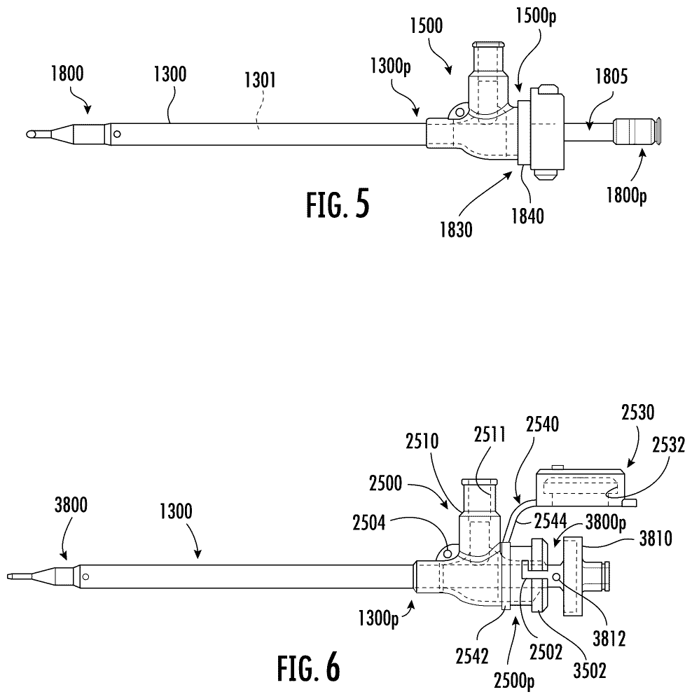

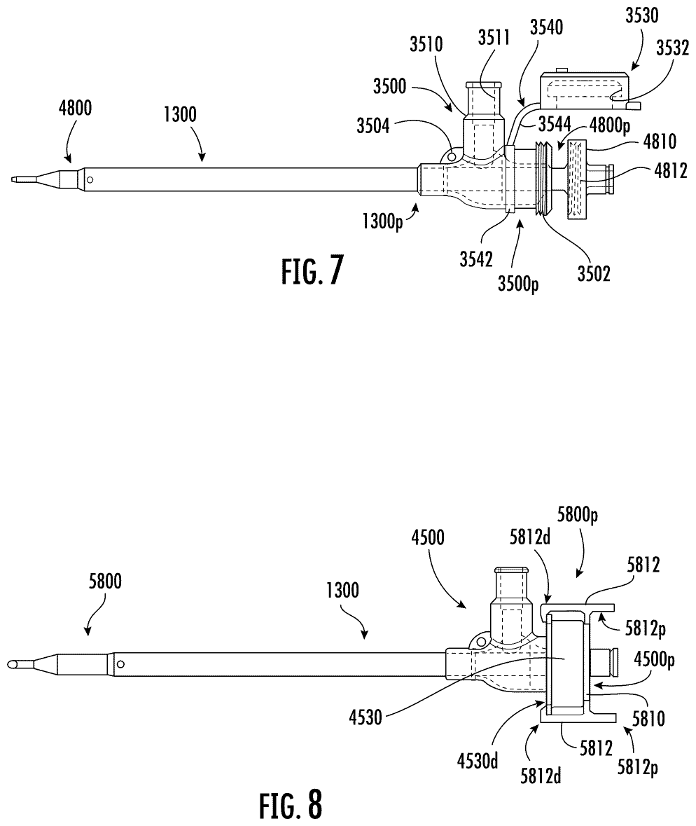

It will be appreciated that engagements between a dilator and an outer sheath 1300 of a fragmenting and/or fragment-removing system formed in accordance with various principles of the present disclosure are not limited to the example of an embodiment illustrated in FIG. 5. Selected examples of alternative embodiments are illustrated in FIG. 6, FIG. 7, FIG. 8, FIG. 9A, FIG. 9B, and FIG. 9C, with similar elements, components, etc., designated with the same reference characters increased by a multiple of 1000, and redundant description omitted, and with elements which are substantially the same generally being indicated by the same reference characters.

In an example of an embodiment illustrated in FIG. 6, an outer sheath 1300 is illustrated as operatively coupled with a fitting 2500 (operatively coupled, for instance, with the proximal end 1300p of the outer sheath 1300) configured to be removably coupled with a dilator 3800. More particularly, the example of an embodiment of a fitting 2500 illustrated in FIG. 6 has a proximal end 2500p with engagement elements configured to releasably couple with mating/corresponding engagement element on a dilator hub 3810 on a proximal end 3800p of the dilator 3800. In the example of an embodiment illustrated in FIG. 6, the engagement element on the fitting 2500 is illustrated as a locking slot 2502 (e.g., an L-shaped slot), configured to mate with a corresponding engagement element on the dilator hub 2810 such as one or more pins 2812. In the example of an embodiment illustrated in FIG. 7, the fitting 3500 has an engagement element in the form of external threads 3502, configured to mate with engagement elements in the form of internal threads 4812 on a dilator hub 4810 on a proximal end 4800p of a dilator 4800. As may be appreciated, the examples of embodiments of a dilator 3800, 4800 illustrated in FIG. 6 and FIG. 7 may be rotated with respect to the respective proximal end 2500p, 3500p of the associated respective fitting 2500, 3500 to engage the associated engagement element 3812, 2502, 4812, 3502. Optionally, a seal 2530, 3530 is coupled to the fitting 2500, 3500 via a tether 2530, 3530 to facilitate coupling of the seal 2530, 3530 with the fitting 2500, 3500 for assembly of the outer sheath 1300 and the inner sheath 1100 in a manner described above and in further detail below. For instance, the tether 2540, 3540 may include a loop 2542, 3542 mounted around the fitting 2500, 3500, and an extension 2544, 3544 extending from the loop 2542, 3542 and coupled (such as welded, glued, interference fit, friction fit, integrally formed (e.g., molded) with, etc.) with the seal 2530, 3530. It will be appreciated that such configuration of a tether may be used with a seal 1430 which is operatively associated with a body 1400 operatively associated with an inner sheath 1100 such as described above. And, various couplings of a seal operatively associated with a body operatively associated with an inner sheath of a fragmenting and/or fragment-removing system formed in accordance with various principles of the present disclosure are applicable to a seal operatively associated with a fitting operatively associated with an outer sheath of the fragmenting and/or fragment-removing system mutatis mutandis. The seal 2530, 3530 may include internal threads 2532 3532 for engaging with the external threads 2502, 3502 on the proximal end 2500p, 3500p of the fitting 2500, 3500 to removably couple the seal 2530, 3530 with respect to the fitting 2500, 3500. Alternative engagement elements for engaging the seal 2530,3530 with respect to the fitting 2500, 3500 (e.g., friction or otherwise, or a Tuohy Borst type configuration, such as described above with respect to the seal 1430) may be used instead, the present disclosure not being limited in this regard. In some embodiments, the seals operatively associated with the body (operatively associated, in turn, with the inner sheath 1100) and with the fitting (operatively associated, in turn, with the outer sheath 1300) are color coded with different colors.

In FIG. 8, an example of an embodiment of a dilator 5800 is illustrated as configured to be releasably coupled to the outer sheath 1300 via a fitting 4500 with one or more engagement elements 5812 in the form of clips 5812. More particularly, the dilator hub 5810 includes one or more clips 5812 movably (e.g., pivotably) coupled with respect to the dilator hub 5810 and having ends 5812d configured to engage with the proximal end 4500p of the fitting 4500. In some embodiments, the ends 5812d of the clips 5812 may be configured as hooks to engage with a shoulder formed on the fitting 4500 and/or the outer sheath 1300. In some aspects, the clips 5812 engage with a seal 4530 (e.g., a distal end 4530d of the seal 4530) removably (e.g., threadedly) coupled with the proximal end 4500p of the fitting 4500. The second ring aids in preventing the seal from being removed. In some aspects, the clips 5812 are pivotably coupled to the dilator hub 5810 and may function as levers, such that depressing the proximal ends 5812p clips 5812 disengages the distal ends 5812d thereof from the fitting 4500/outer sheath 1300/seal 4530 for removal therefrom. It will be appreciated that the present disclosure encompasses a reverse configuration as well, with levers formed on the fitting 4500/outer sheath 1300/seal 4530 engaging with the dilator 5800/dilator hub 5810 (e.g., with a shoulder formed thereon).

It will be appreciated that the fitting 1500, 2500, 3500, 4500 which is operatively associated with the outer sheath 1300 need not be limited to the T-shape connector configuration illustrated in FIGS. 1-3, FIGS. 4A-4C, or FIGS. 5-8. For instance, a fitting 5500 with a curved sidearm 5510 may be operatively associated with a proximal end 1300p of an outer sheath 1300, such as illustrated in FIG. 9. More particularly, the curved sidearm 5510 of the example of an embodiment of a fitting 5500 illustrated in FIG. 9 has a lumen 5511 defined therethrough in communication with the lumen 5501 defined generally axially through main body/housing 5502 of the fitting 5500 and in which the proximal end 1300p of the outer sheath 1300 is inserted. As may be appreciated, like the lumens 1501 and 1511 of the above-described fitting 1500, the lumens 5501 and 5511 of the fitting 5500 are in fluid communication with the lumen 1301 of the outer sheath 1300. As such, elements, members, tools, instruments, materials, fluids, etc., flow between the lumens 1301, 5501, and 5511. As such, a dilator 1800, 2800, 3800, 4800, 5800 such as described above may be inserted into the curved sidearm 5510 of the fitting 5500 to extend into the lumen 5511 and into the lumen 5501 of the fitting 5500, and into the lumen 1301 of the outer sheath 1300. The distal end of the dilator may be extended distally past the distal end of the outer sheath 1300 in a manner to facilitate navigation of the outer sheath 1300 to the treatment site, such as in a manner known to those of ordinary skill in the art.

Similarly, it will be appreciated that a body coupled to a proximal end of an inner sheath of a fragmenting and/or fragment-removing system formed in accordance with various principles of the present disclosure may have other configurations. For instance, instead of a body 1400 with two side arms, such as illustrated in FIG. 1, FIG. 2, and FIG. 3, a body with a single side arm may be associated with an inner sheath of a fragmenting and/or fragment-removing system formed in accordance with various principles of the present disclosure. For instance, a fitting 5500 such as illustrated in FIG. 9 (for operative association with a proximal end of an outer sheath), may be operatively associated with a proximal end of an inner sheath of a fragmenting and/or fragment-removing system formed in accordance with various principles of the present disclosure, with any appropriate modifications being made to accommodate the use of the inner sheath in accordance with various principles of the present disclosure.

Once the distal end of an outer sheath of a fragmenting and/or fragment-removing system formed in accordance with various principles of the present disclosure has been guided to a treatment site, such as with the assistance of a dilator and/or guidewire, the dilator and/or guidewire may be removed and the inner sheath may be inserted into the outer sheath. Optionally, a tubular elongate member and an optional biasing member may also be inserted into the outer sheath. The distal ends of the inner sheath, the tubular elongate member, and/or the biasing member may be extended distal to the distal end of the outer sheath and into the treatment site.

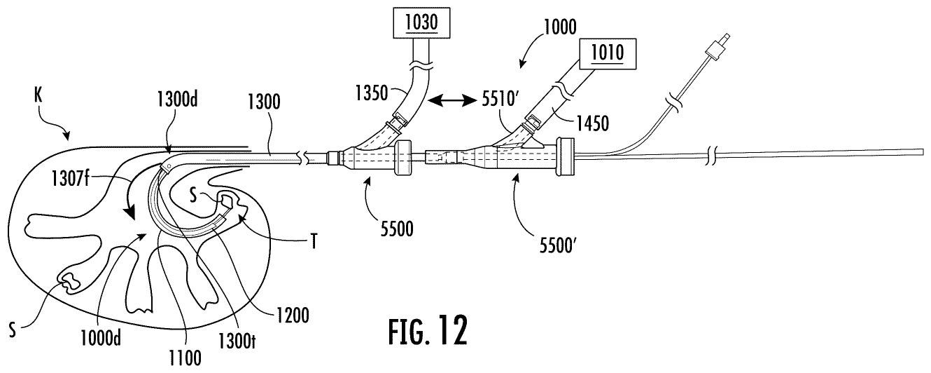

In accordance with various principles of the present disclosure, an inner sheath may be extended into the outer sheath through a hole in a seal on a proximal end of a fitting operatively associated with a proximal end of the outer sheath. An example of an embodiment of a seal 5530 which may be fitted on a proximal end 5500p of a fitting 5500 such as illustrated in FIG. 9 is illustrated in FIG. 10A and FIG. 10B. As may be appreciated, the illustrated example of an embodiment of a seal 5530 has an aperture or hole 5531 configured to receive an element to be inserted into the lumen 5501 of the fitting 5500. As operatively associated with a fitting 5500 which is operatively associated with an outer sheath 5300, such as illustrated in FIG. 9, insertion of an element into the hole 5531 of the seal 5530 provides access to the lumen 1301 of the outer sheath 1300. An inner sheath 1100 such as illustrated in any of FIG. 1, FIG. 2, FIG. 3, or FIGS. 4A-4C may thus be inserted into the hole 5531 of the seal 5530 and thereby into the lumen 1301 of the outer sheath 1300 for advancement to a treatment site. It will be appreciated that the seal 5530 illustrated in FIG. 10A and FIG. 10B may also represent a seal configuration suitable for operative association with a proximal end of a body 1400 as illustrated in any of FIG. 1, FIG. 2, FIG. 3, or FIGS. 4A-4C, and/or a fitting 5500 such as illustrated in FIG. 9 configured for operative association with an inner sheath (e.g., with a proximal end of an inner sheath). In such instance, the hole 5531 of the seal 5530 provides access to the lumen 5501 of the fitting 5500 and also to the lumen 1101 of the inner sheath 1100 for advancement through the inner sheath to a treatment site. It will be appreciated that the material of the seal 5530 may be generally elastomeric to provide a fluid tight seal with an element, member, instrument, tool, etc., inserted into the hole 5531 thereof, such as in a manner known those of ordinary skill in the art. Furthermore, it will be appreciated that reference may be made herein to element, member, instrument, tool, etc., interchangeably, without intent to limit.

It will be appreciated, as described above, that a tubular elongate member may be inserted into a proximal end of a body operatively associated with a proximal end of the inner sheath. Optionally, as described above, a biasing member and/or a separate sensor may also be inserted into the proximal end of the body. In such embodiment, a seal with two holes for independent insertion of a tubular elongate member as well as a biasing member may be operatively associated with the proximal end of a body or fitting operatively associated with a proximal end of the inner sheath. An example of an embodiment of a seal 5530′ with a first hole 5531′ and a second hole 5533′ and which may be operatively associated with an inner sheath formed in accordance with various principles of the present disclosure is illustrated in FIG. 11A and FIG. 11B. The illustrated example of an embodiment of a seal 5530 or a seal 5530′ may be operatively associated with (e.g., mounted on) the proximal end 1400p of a body 1400 such as illustrated in any of FIG. 1, FIG. 2, FIG. 3, or FIGS. 4A-4C and/or the proximal end 1500p of a fitting 1500 such as illustrated in any of FIG. 1, FIG. 2, FIG. 3, or FIGS. 4A-4C, and/or the proximal end 5500p of a fitting 5500 such as illustrated in FIG. 9.

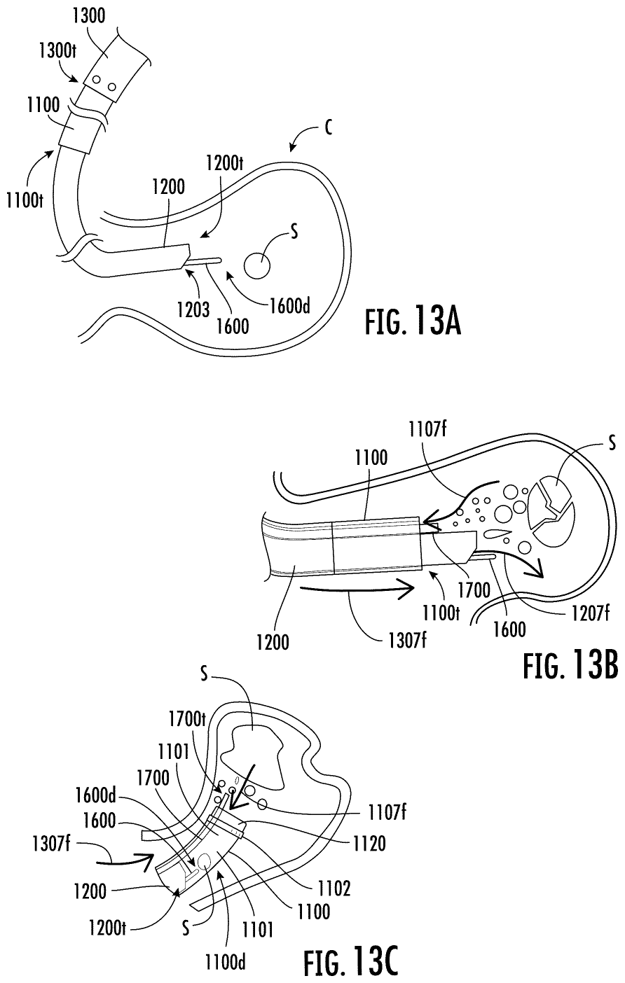

Just as various configurations of seals may be used on either of the inner sheath or the outer sheath of a fragmenting and/or fragment-removing system formed in accordance with various principles of the present disclosure, various configurations of bodies and/or fittings may be operatively associated with either of the inner sheath or the outer sheath. For instance, as illustrated in FIG. 12, a body 5500′ operatively associated with an inner sheath 1100 may have a configuration similar to that of the fitting 5500 illustrated in FIG. 9. The fitting 5500 operatively associated with the outer sheath 1300 of the example of an embodiment of a fragmenting and/or fragment-removing system 1000 illustrated in FIG. 12 has a similar, albeit optionally smaller, configuration as that of the body 5500′. As may be appreciated, the example of an embodiment of a body 5500′ illustrated in FIG. 12 may be considered to be similar to the body 1400 illustrated in FIG. 1, FIG. 2, FIG. 3, or FIGS. 4A-4C, except the body 5500′ has only one side arm 5510′, in contrast with the two sidearms 1410, 1420 of the body 1400. The single sidearm may be fluidly coupled with the outlet channel 1107 of the fragmenting and/or fragment-removing system 1000 (e.g., and fluidly coupled with a suction source 1010 such as illustrated in FIG. 1), and the tubular elongate member 1200 and optional biasing member 1700 and optional separate sensor 1710 may be inserted through the proximal end of the body (such as in a manner described above with reference to the example of an embodiment illustrated in FIGS. 4A-4C. It will be appreciated that other components, devices, elements, etc., illustrated in FIG. 12 similar to or the same as in previously discussed figures are indicated with the same reference characters, and reference is made to the above-descriptions thereof for the sake of brevity. It will be appreciated that other configurations of a body with a single arm, like the body 5500′ illustrated in FIG. 12, may be associated with an inner sheath 1100. For instance, instead of the single arm being curved as illustrated in FIG. 12, the single arm may be straighter, similar to the first sidearm 1410 of the body 1400 illustrated in FIG. 1, FIG. 2, FIG. 3 and FIGS. 4A-4C. With an embodiment of a body having a single sidearm, Ta dilator (if used) would be inserted into the inner sheath 1100 through the proximal end of the body, such as illustrated in FIGS. 4A-4C. The associated seal may have a single hole (e.g., like the seal 1430 illustrated in FIG. 1C) or two holes (e.g., like the seal 5530′ illustrated in FIG. 11A and FIG. 11B). As may be appreciated, other configurations of bodies or fittings may be used in a fragmenting and/or fragment-removing system 1000 of the present disclosure, and such configurations may be mixed and matched between the inner sheath 1100 and the outer sheath 1300, the present disclosure not being limited in this regard.

As noted above, in some aspects, once a dilator is assembled with an outer sheath and/or an inner sheath the unit can be inserted into the body to the treatment site (e.g., up to the UPJ, such as illustrated in FIG. 1), such as over an already-in-place guidewire. As described above, an inner sheath of a fragmenting and/or fragment-removing system of the present disclosure can be assembled with a tubular elongate member (e.g., a medical scope) and an optional biasing member extending therethrough. The tubular elongate member may define a lumen through which a proximal inlet may be defined, and/or through which a fragmenting device (such as a laser) may be advanced to a treatment site. The distal ends of the tubular elongate member and/or the biasing member are advanced to the distal end of the inner sheath, and may provide support and/or steering capability to the inner sheath. The inner sheath, with or without the tubular elongate member and the biasing member already assembled therein, are inserted into and advanced distally through the outer sheath, and distal to the distal end of the outer sheath to the treatment site).

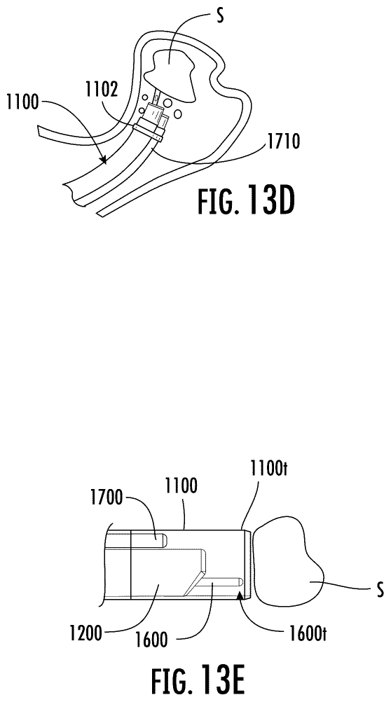

Use of a fragmenting and/or fragment-removing system 1000 formed in accordance with various principles of the present disclosure will now be described with reference to a kidney K as a treatment site T, and a stone S as a bodily mass to be removed from the treatment site T, such as illustrated in FIG. 1, FIG. 2, FIG. 3, FIG. 12, and FIG. 13. Moreover, reference may be made to a tubular elongate member in the form of an ureteroscope for the sake of convenience and without intent to limit. However, principles of the present disclosure are applicable to other anatomical sites/structures, and/or other types of bodily masses, and/or other types of tubular elongate members, as may be appreciated by those of ordinary skill in the art.

In the example of an embodiment of a fragmenting and/or fragment-removing system 1000 illustrated in FIG. 13, the main body 1402′ of the body 1400′ coupled to the proximal end 1100p of the inner sheath 1100 is illustrated as longer than as illustrated in FIG. 1, FIG. 2, and FIG. 3. In some aspects, the body 1400′ is selected to have a main body 1402′ with a length sufficient to allow the flexible distalmost end 1200t of the ureteroscope 1200 to be supported within the body 1400 when withdrawn proximally to clear/withdraw clogs from within the inner sheath 1100 and/or to remove fragments by “virtual basketing” (without a traditional basketing device) as described in further detail below. In some aspects, the distalmost end 1200t of the ureteroscope 1200 is not withdrawn completely from the body 1400′, such as to allow the ureteroscope 1200 to be quickly reinserted after the clog/fragments has been suctioned through the first sidearm 1410′.

With the distal end 1000d of a fragmenting and/or fragment-removing system 1000 formed in accordance with various principles of the present disclosure at a treatment site T, such as illustrated in FIG. 13, the initial placement of the fragmenting and/or fragment-removing system 1000 may be considered to be complete, or at least ready for initial use. It will be appreciated that the outer sheath 1300 need not be distally advanced beyond an entry point to the treatment site T. For instance, in the example of an embodiment of a treatment site T illustrated in FIG. 13, the distal end 1300d of the outer sheath 1300 is inserted up to the UPJ of a kidney K. The inner sheath 1100 and/or the tubular elongate member 1200 are inserted distally beyond the distalmost end 1300t of the outer sheath 1300 to a treatment site T of interest (in the example of an embodiment illustrated in FIG. 13, a stone S). A fragmenting device 1600 (such as a laser) may be advanced to the stone S and activated to fragment, dust, reduce, etc., the stone S, such as in a manner known those of ordinary skill in the art, the present disclosure not being limited in this regard. The fragmenting device 1600 may be activated in any of a variety of manners, such as via a foot petal, the present disclosure not being limited in this regard.

The tubular elongate member 1200 may be configured to be navigated, either when distally extended out of the inner sheath 1100, or when still within the inner sheath 1100, such as optionally also deflecting a passive inner sheath 1100 (such as described above). As may be appreciated, such as with reference to FIG. 13, the tubular elongate member 1200 (e.g., ureteroscope 1200), may be rotated and/or deflected to reach a stone S located in any of the poles of the kidney K. For instance, in the example of an embodiment illustrated in FIG. 13, the distal end 1200d of the ureteroscope 1200 is positioned at the lower pole LP of the kidney K, such as to diagnose, assess, locate, etc., the targeted stone S. The medical professional operating the fragmenting and/or fragment-removing system 1000 has the option to reposition the stone S to the upper pole UP of the kidney K, or to remove the stone S using a basket, or to fragment (e.g., to laser treat) the stone in the lower pole LP.

A fragmenting device 1600, such as a laser may be extended through the working channel 1203 of the ureteroscope 1200 (see, e.g., FIG. 1B) to fragment and/or dust the stone S.

As illustrated in FIG. 13A, showing the detail area 13B of FIG. 13 (though not with the members 1100, 1200, 1300, 1600, 1700, etc., of the fragmenting and/or fragment-removing system 1000 in the relative positions illustrated in FIG. 13), the distalmost end 1200t of the ureteroscope 1200 may be extended distally out of (and distal to) the distalmost end 1100t of the inner sheath 1100 and to a stone S in a calyx C of the kidney K (e.g., in a lower pole LP of the kidney K). A fragmenting device 1600 may be extended out of the working channel 1203 of the medical scope 1200 (such as illustrated in FIG. 1A and FIG. 1B, as well as in the detail view of FIG. 13A) and closer to the stone S. The fragmenting device 1600 may then be activated to fragment, dust, etc., the stone S, such as depending on the type of device used as a fragmenting device 1600, and/or the nature of the energy applied by the fragmenting device 1600 (e.g., a Rotablator™ Rotational Atherectomy System, sold by Boston Scientific Inc, or a Swiss LithoClast™ lithotripter, also sold by Boston Scientific, Inc.). For instance, in the illustrated example of an embodiment, the fragmenting device 1600 may be a laser fiber 1600 extended distally out of the working channel 1203 of the ureteroscope 1200, and the stone S is lased to fragments or dust (e.g., depending on the characteristics of the energy applied to the laser, such as known those of ordinary skill in the art, the present disclosure not being limited in this regard).

In accordance with various principles of the present disclosure, once fragmentation, dusting, etc., of a bodily mass (e.g., stone S) by a fragmenting device has commenced, it may be desirable to irrigate and/or aspirate the treatment site, such as to move/remove particles from the treatment site (and/or an optional visualization device, such as a camera of a medical scope such as a ureteroscope); and/or to regulate temperature and/or pressure at the treatment site; etc. In the example of an embodiment of a fragmenting and/or fragment-removing system 1000 illustrated in FIG. 13, an aspiration connection tube 1450 may be fluidly coupled between a suction source 1010 and the outlet sidearm 1410′ of the body 1400′ which is in fluid commination with the inner sheath 1100; and/or the ureteroscope 1200 may be fluidly coupled with a first fluid source 1020; and/or an inlet irrigation connection tube 1550 may be fluidly coupled between a second fluid source 1030 and the sidearm 1510 of the fitting 1500 which is in fluid commination with the lumen 1301 within the outer sheath 1300. In accordance with various principles of the present disclosure, a sensor may be associated with the fragmenting and/or fragment-removing system 1000 capable of sensing one or more conditions at the treatment site T including temperature and/or pressure. For instance, a flow meter 1452 may be mounted proximally on the suction connector tube 1450 to detect a clog therein. As described above, a pressure and/or temperature sensor may be included with the biasing member 1700 as a sensor element 1702 thereof (such as illustrated in FIG. 1A and FIG. 13B) or as a separate member 1710 of the fragmenting and/or fragment-removing system 1000 (such as illustrated in FIG. 13D). Additionally or alternatively, a sensor may be provided to visualize conditions at the treatment site. In some embodiments, the tubular elongate member 1200 is a ureteroscope (e.g., a flexible ureteroscope) provided with a visualization element 1210 (such as a camera, such as illustrated in FIG. 1A and FIG. 1B). Information acquired by any or all of the sensors of the fragmenting and/or fragment-removing system 1000 may be used to control the relative positions of the various members 1100, 1200, 1300, 1600, 1700, etc., of a fragmenting and/or fragment-removing system 1000 formed in accordance with various principles of the present disclosure, and/or any or all of the suction source 1010, the first fluid source 1020, or the second fluid source 1030, such as discussed in further detail below.

In some aspects, the inner sheath 1101 is advanced distally to bring the distalmost end 1100t thereof closer to the distalmost end 1200t of the ureteroscope 1200 after fragmenting (e.g., lasing) a stone S, such as illustrated in FIG. 13 and in FIG. 13B (showing Detail Area 13B in FIG. 13B in further detail), to allow stone fragments to be aspirated out of the kidney K via the outlet 1107 defined through the inner sheath 1100. In some aspects, the inner sheath 1101 is advanced to the site at which a stone S is located before fragmenting the stone S. For instance, the inner sheath 1100 may be advanced to the stone S before lasing the stone S, such as to aspirate dust/fragments/elevated temperatures simultaneously during lasing to clear the field of view and the target site of fragments and/or to regulate temperature and/or pressure at the site of lasing. In some aspects, stone fragments may be sized (e.g., to fit into the inner sheath 1100) using the edge or distal end 1100d of the inner sheath 1100 as an indicator.

In some aspects, during initial fragmenting of a stone S, it may be desirable for the outlet 1107 defined by the inner sheath 1100 to be closer to the distal end 1600d of the fragmenting device 1600 than as illustrated in FIG. 13A, such as when lasing with a fragmenting device 1600 in the form of a laser fiber. For instance, as illustrated in FIG. 13B, the inner sheath 1100 and the ureteroscope 1200 may be advanced distally out of the outer sheath 1300 with the respective distal ends 1100d, 1200d thereof advancing generally adjacent each other to be introduced together into the lower pole LP of the kidney K and to a stone S. If provided, a radiopaque marker 1102 along the distal end 1100d of the inner sheath 1100 may facilitate guiding of the inner sheath 1100 to a stone S. The inner sheath 1100 may be a generally passive elongate element steered by the steerable distal end 1200t of the ureteroscope 1200 positioned therein. Alternatively, the distal end 1200d of the tubular elongate member 1200 may be advanced to the stone S first, and the inner sheath 1100 may then be advanced over the tubular elongate member 1200 for the distal end 1100d of the inner sheath 1100 to reach a position as illustrated in FIG. 13B. The distal inlet channel 1207 defined by the working channel 1203 of the ureteroscope 1200 (see, e.g., FIG. 1B) may be used for irrigation of the treatment site T (e.g., during lasing with the fragmenting device 1600), and/or to flush, clean, move, group, and/or disperse dust/fragments. If a visualization device/element 1210 is provided, such as at the distal end 1200d of the ureteroscope 1200, the distal inlet channel 1207 (e.g., proximal inlet flow 1307f therefrom into the treatment site T) may clear the field of view of the visualization element 1210. In the configuration illustrated in FIG. 13B, the outlet channel 1107, defined within the inner sheath 1107 and between the inner surface 1103 of the inner sheath 1100 and the outer surface 1205 of the ureteroscope 1200, is positioned proximal to the distalmost end 1200t of the ureteroscope 1200. The proximal inlet channel 1307 defined within the outer sheath 1300 may be used to supply an inlet flow 1307f to replenish fluid that has been aspirated out of the treatment site T.

Even with a biasing member 1700 maximizing the spacing between the inner surface 1103 of the inner sheath 1100 and the outer surface 1205 of the ureteroscope 1200, in some cases, the outlet channel 1107 may allow only small fragments and elevated temperatures to be suctioned into the outlet channel 1107 (e.g., along suction flow 1107f) to be aspirated out of the treatment site T. Some fragments of a stone S may be too large to readily pass from the treatment site T and out through the outlet channel 1107. In some aspects, the inner sheath 1100 is distally advanced a short distance distal to the distalmost end 1200t of the tubular elongate member 1200 and/or the distalmost end 1200t of the tubular elongate member 1200 is proximally retracted a short distance proximal to the distalmost end 1100t of the inner sheath 1100 to form a funnel 1120 within the distal end 1100d of the inner sheath 1100, as illustrated in FIG. 13C. The funnel 1120 provides a wider area within the inner sheath 1100 to allow fragments of stones S to be guided with the suction flow 1107f towards and into the funnel 1120 defined within the inner sheath 1100 and between the distalmost end 1100t of the inner sheath 1100 and the distalmost end 1200t of the tubular elongate member 1200. Suction applied via the outlet channel 1107 can be used to suction a stone S to the distalmost end 1100t of the inner sheath 1100, or the inner sheath 1100 may be extended to the stone S. If provided, a radiopaque marker 1102 along the distal end 1100d of the inner sheath 1100 may facilitate positioning of the inner sheath 1100 with respect to a stone S. The fragmenting device 1600 may be a laser fiber which lases stones S, or fragments thereof, within the funnel 1120, optionally “popcorning” the stones S and fragments thereof against one another and/or against the inner surface 1103 of the inner sheath 1100. At least a distal portion, and optionally the entire length of the inner sheath may be formed of a laser-resistant type material. In some aspects, the laser-type material is provided as a liner. In some aspects, at least the inner sheath 1100 is reinforced by a coil or braid (e.g., stainless steel, etc.). Optionally, the distal end 1600d of the fragmenting device 1600 may be extended distal to the distalmost end 1100t of the inner sheath 1100 to laser/fragment/dust stones S outside the funnel 1120. As irrigation (e.g., proximal inlet flow 1307f) and suction (e.g., outlet flow 1107f) are applied, large and small fragments of stones that will fit within the funnel 1120 will be suctioned into the inner sheath 1101 and fragmented therein, and then suctioned out of the treatment site T via the outlet channel 1107. It will be appreciated that the biasing member 1700 remains in place within the lumen 1101 of the inner sheath 1100, maximizing a spacing between the inner sheath 1100 and the tubular elongate member 1200 to maximize the size of a stone fragment which may be suctioned out of the treatment site T via the outlet channel 1107. In some aspects, stones S may be laser pulverized to fine dust while contained within the funnel 1120 defined within the distal end 1100d of the inner sheath 1100, and aspirated out, via the outlet channel 1107, substantially simultaneously as the stone S is being lasered. Even with the increased volume provided by a funnel 1120 such as described above, some larger fragments may not be able to fit inside the distal end 1100d of the inner sheath 1100. In some aspects, larger fragments may be laser pulverized while held against the distalmost end 1300t of the inner sheath 1100.

In some aspects, larger stones S may be “virtually” basketed with a fragmenting and/or fragment-removing system 1000 such as formed in accordance with various principles of the present disclosure. For instance, as illustrated in FIG. 13E, the distalmost end 1600t of the fragmenting device 1600 as well as the distalmost end 1700t may be withdrawn proximal to the distalmost end 1100t of the inner sheath 1100, and thus within the lumen 1101 of the inner sheath 1100. Alternatively, the distalmost end 1100t of the inner sheath 1100 may be positioned immediately adjacent a stone S too large to fit within the lumen 1101 of the inner sheath 1100. The distalmost end 1100t of the inner sheath 1100 may be used as an indicator for sizing the stone to fit into the inner sheath 1100. The laser fiber 1600 may then be extended to the stone S to fragment or dust the stone S further to fit into the lumen 1101 of the inner sheath 1100. The laser fiber 1600 may further break down stones S capable of fitting in a funnel 1120 such as described above but not capable of being suctioned through the outlet channel 1107. For instance, the laser fiber 1600 may lase stones S within the distal end 1100d of the inner sheath 1100 with the ureteroscope 1200 proximally retracted within the inner sheath 1100 to allow a larger space for the stone S. Virtual basketing may be performed to remove stones S too large to fit through the outlet channel 1107. For instance, stone fragments, clogs, and/or debris may be suctioned into the inner sheath 1100 and against the distalmost end 1200t of the ureteroscope 1200. The laser fiber 1600 may be extended distal to ureteroscope 1200, such as to be in the field of view of the visualization element 1210 thereof (and still inside the inner sheath 1100). Suction force applied by the outlet channel 1107 moves the fragments towards the laser fiber 1600 to facilitate lasing, as well as to minimize laser retropulsion of the fragment out of the inner sheath 1100. Moreover, as the inner sheath 1100 contains/shields the heat and dust within the funnel 1170, suction through the outlet channel 1107 transports not only the fluid with laser-pulverized stone debris, but also elevated temperatures (e.g., caused by lasing) proximally out of the treatment site T.

Upon proximal withdrawal of the ureteroscope 1200 through the inner sheath 1100, the debris will be suctioned proximally to follow the ureteroscope 1200 towards the proximal end 100p of the fragmenting and/or fragment-removing system 100. Once the ureteroscope 1200 has reached just proximal to the first sidearm 1410′ of the body 1400′ (through which suction is applied into the inner sheath 1100), the medical professional may view on the image/display monitor of the ureteroscope 1200 (e.g., operatively coupled with the visualization element 1210), that the debris has been suctioned away from the distalmost end 1200t of the ureteroscope 1200 and out of the fragmenting and/or fragment-removing system 1000 via the first sidearm 1410′ of the body 1400′. At this point, the distal end of the distal inlet channel 1207 is proximal to the distal end of the proximal inlet channel 1307. Once the thus virtually-basketed fragments have been removed from the inner sheath 1100, the medical professional may advance the distalmost end 1200t of the ureteroscope 1200 back to the distal end 1100d of the inner sheath 1100 for additional stone removal. It will be appreciated that in some instances unclogging of the inner sheath 1100 (of stone dust or larger fragments) may only require the distalmost end 1200t of the ureteroscope 1200 to be withdrawn a shorter distance than all the way to the first sidearm 1410′ of the body 1400′.

In some embodiments, the body 1400′ is formed of a clear or transparent material, such as to facilitate viewing of the position of the tubular elongate member 1200 therein. Additionally or alternatively, the body 1400′ is molded or stamped or heat stamped or otherwise provided with a limit indicator or marking 1404′ and optional directional indicia 1406′ indicating a position which the distalmost end 1200t of the ureteroscope 1200 reaches when further proximal withdrawal thereof may be stopped. Additionally or alternatively, the medical professional may use the position of the first sidearm 1410′ as the limit indicator. Additionally or alternatively, the limit indicator may be molded as an internal taper step 1408′. Additionally or alternatively, the medical professional may simply use the visualization element 1210 and associated system of the ureteroscope 1200 to image the location of the distalmost end 1200t of the ureteroscope 1200 to prevent total removal of the ureteroscope 1200 from the body 1400′. Additionally or alternatively, the main body 1402′ is made in two parts. In some aspects, the distal part of the two-part main body 1402′ may be more rigid than the proximal part, and may include a seal 1430 such as described above. Optionally, the two parts of the main body 1402′ are telescopically coupled (such as in a manner known those of ordinary skill in the art).

As may be appreciated, various of the above-described sensors may be used during use of a fragmenting and/or fragment-removing system 1000 formed in accordance with various principles of the present disclosure to facilitate fragmenting and/or removal of fragments from a treatment site T. For instance, the above-described visualization element 1210 may be used to view a stone S during fragmenting and/or removal, and/or if too many fragments are detected, and/or if the stone S is too large to fit within the lumen 1101 of the inner sheath 1100, and/or if further irrigation is required to clear the treatment site T, and/or the position of the distalmost end 1100t of the inner sheath 1100 (such as relative to the tubular elongate member 1200 and/or the fragmenting device 1600 and/or the biasing member 1700), etc. As may be appreciated, various adjustments to one or more elements of the fragmenting and/or fragment-removing system 1000 and/or the suction and/or irrigation supplied thereto may be made based on the information obtained by the visualization element 1210.

In embodiments in which a temperature and/or pressure sensor is provided (e.g., as a sensor element 1702 of the biasing member 1700, or as a separate, independent element 1710), the sensor may be operatively coupled with a fluid management system 1040, such as illustrated in FIG. 1, or another type of controller such as known those of ordinary skill in the art. Optionally, the ureteroscope 1200 may also be operatively coupled with the fluid management system 1040 (to simplify the drawings, schematic connections of the fluid management system 1040 are not illustrated, such connections being readily understandable by to those of ordinary skill in the art). Various controls (e.g., buttons, dials, etc.) on the scope handle can be used to prime, flush, accelerate, start, stop, etc., irrigation fluid supplied through the working channel thereof and thus the distal inlet channel 1207 of the fragmenting and/or fragment-removing system 1000.

The fluid management system 1040 (or other controller) may be used to control the fluid dynamics of the fragmenting and/or fragment-removing system 1000 and/or within the treatment site T. For instance, the fluid management system 1040 may be used to control the magnitude of the vacuum applied by the suction source 1010, and/or fluid delivered by one or both fluid sources 1020, 1030, such as by controlling the speed of pumps operating one or more of the suction source 1010 or fluid source 1020 or fluid source 1030. For example, a preferred renal pressure operating range and/or renal temperature operating range may be set and/or adjusted by the medical professional (optionally with the use of the fluid management system 1040) based on information obtained by the sensor element 1702 or separate sensor 1710. For instance, when the renal pressure is too low, the fluid management system 1040 may increase the inlet/irrigation flow to quickly to refill and balance to the appropriated pressure range. When renal pressure is elevated, the fluid management system 1040 may increase the suction to exchange out the excessive fluid to the appropriate pressure range. When the renal temperature is elevated and the pressure is at an acceptable range, the fluid management system 1040 may increase both the inlet and outlet flow, such as to have the same flow rate to quickly exchange out the excess heat. When the renal temperature and the pressure are both elevated, the fluid management system 1040 may increase both the inlet flow and the outlet flow. Optionally, the fluid management system 1040 may increase suction more than the irrigation rate to quickly exchange out the excess heat and pressure. When the renal temperature is elevated, and the pressure is low, the fluid management system 1040 may increase both the inlet flow and the outlet flow. Optionally, the irrigation rate may be increased more than the suction rate to quickly exchange out the excess heat and replenish the fluid. It will be appreciated that the arrangement of a fluid management system 1040 as illustrated in FIG. 13 is schematic, the present disclosure not being limited in this regard. Moreover, it will be appreciated that the above descriptions of use may be applied to any of the various combinations, subcombinations, arrangements, etc., of devices, elements, members, etc., of a fragmenting and/or fragment-removing system formed in accordance with various principles of the present disclosure.

In some embodiments, a governing hole 1414′ may be provided on the first sidearm 1410′, such as to allow manual control of the outlet flow 1707f through the outlet channel 1107 defined through the inner sheath 1100. In a manual use/control mode, covering of the governing hole 1414′ with a finger/thumb (e.g., of the hand holding the body 1400′) will increase the negative pressure to the outlet channel 1707 defined through the inner sheath 1100, such as by preventing suction from being lost. Uncovering the governing hole 1414′ will decrease the negative pressure to the outlet channel 1707 defined through the inner sheath 1100. An uncovered governing hole 1414′ configured as a passive drain (e.g., without negative pressure or a suction source 1010 fluidly coupled thereto) may enable proximal fluid drainage proximally through the inner sheath 1100. Partially covering the governing hole 1414′ can control the magnitude of the suction. Typically, a governing hole 1414′ need not be present when a fluid management system 1040 is used to regulate the fragmenting and/or fragment-removing system 1000 (e.g., to regulate the flow rate/fluid volume flow into and out of the treatment site T, thus regulating the pressure and temperature at the treatment site T).

It will be appreciated that various features of one example of an embodiment of a medical device described herein may be present in others of the examples of embodiments described herein, whether or not explicitly stated or described. For purposes of clarity, not all components having the same reference number are numbered. It will further be appreciated that the various features described herein may be used singly or in any combination thereof. Therefore, the present invention is not limited to only the combination of features described with respect to the embodiments specifically described herein. Moreover, it will be appreciated that various operations of an above-described device or system may be performed by others of the above-described devices or systems.

Although embodiments of the present disclosure may be described with specific reference to medical devices and systems and procedures for treating the urinary tract, it should be appreciated that such medical devices and methods may be used to treat tissues of the gastrointestinal system, abdominal cavity, digestive system, urinary tract, reproductive tract, respiratory system, cardiovascular system, circulatory system, and the like. Moreover, the medical devices, instruments, tools, etc. of the present disclosure are not limited, and may include a variety of medical devices, instruments, tools, etc., for accessing body passageways, including, for example, ureteroscopes, duodenoscopes, catheters, bronchoscopes, colonoscopes, arthroscopes, cholangioscopes, cystoscopes, hysteroscopes, and the like. Such devices, instruments, tools, etc., may be disposable and for single use only, or sterilizable and reusable, the present disclosure not being limited in this regard.

Although embodiments of the present disclosure may be described with specific reference to medical devices and systems (e.g., endoscopic devices, accessory tools, and/or guidewires inserted near or through urinary tract, etc.), it should be appreciated that such medical devices and systems may be used in a variety of medical procedures for navigating one or more devices through ductal, luminal, vascular, or body lumen anatomies. The disclosed medical devices and systems may be inserted via different access points and approaches, e.g., percutaneously, endoscopically, laparoscopically, or combinations thereof, the present disclosure not being limited in this regard.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for removing fragmented remains of a bodily mass from an anatomical site within a patient, said system comprising:
a first sheath defining a first lumen therethrough and having an inner surface and an outer surface;
a tubular elongate member extending through the first lumen, said tubular elongate member defining a distal inlet channel therethrough and having an inner surface and an outer surface; and
a biasing member within said first lumen maintaining spacing between the inner surface of said first sheath and said outer surface of said tubular elongate member;
wherein:
said first lumen is fluidly couplable with a suction source to define an outlet channel between the inner surface of said first sheath and said outer surface of said tubular elongate member; and
said biasing member is insertable into said first lumen independently of said tubular elongate member.

2. The system of claim 1, wherein said first sheath, said tubular elongate member, and said biasing member are movable with respect to one another to adjust the relative positions of distalmost ends thereof with respect to one another.

3. The system of claim 2, wherein said first sheath has a distalmost end advanceable distal to a distalmost end of said tubular elongate member to create a funnel within a distal end of said first sheath within said first lumen and between the distalmost end of said first sheath and the distalmost end of said tubular elongate member.

4. The system of claim 1, wherein said biasing member includes a sensor.

5. The system of claim 1, further comprising a sensor insertable into said first lumen independently of said tubular elongate member and said biasing member.

6. The system of claim 1, wherein said tubular elongate member is a medical scope with a working channel defining the distal inlet channel.

7. The system of claim 1, further comprising a second sheath defining a second lumen therethrough and having an inner surface and an outer surface, said first sheath positionable within the second lumen defined through said second sheath, wherein a proximal inlet channel is defined between the inner surface of said second sheath and the outer surface of said first sheath.

8. The system of claim 1, further comprising a fragmenting device extendable through said tubular elongate member.

9. The system of claim 8, wherein said fragmenting device is movable with respect to said first sheath and said tubular elongate member between a position distal to a distalmost end of said first sheath and a position proximal to the distalmost end of said first sheath and within the first lumen.

10. The system of claim 1, further comprising a fluid management system operatively associated with at least one of the first lumen or the distal inlet channel to control pressure and/or temperature conditions at the anatomical site.

11. A system for removing fragments of a bodily mass from an anatomical site, said system comprising:
an outer sheath defining an outer system lumen therethrough and having an inner surface and an outer surface;
an inner sheath defining an inner system lumen therethrough and having an inner surface and an outer surface; and
a tubular elongate member extending through the inner system lumen and defining a distal inlet channel therethrough fluidly couplable with a fluid source to irrigate the anatomical site;
wherein:
a space defined within the outer system lumen and between the inner surface of said outer sheath and the outer surface of said inner sheath defines a proximal inlet channel fluidly couplable with a second fluid source to irrigate the anatomical site; and
a space defined within the inner system lumen and between the inner surface of said inner sheath and the outer surface of said tubular elongate member defines an outlet channel couplable with a suction source to aspirate a bodily mass or fragments thereof from the anatomical site.

12. The system of claim 11, further comprising a fragmenting device extendable through the tubular elongate member.

13. The system of claim 11, further comprising a dilator insertable within at least one of the outer sheath or the inner sheath.

14. The system of claim 11, further comprising a sensor operatively associated with one of said inner sheath or said tubular elongate member, or insertable separate from said inner sheath and said tubular elongate member.

15. The system of claim 11, further comprising a fluid management system operatively associated with at least one of said proximal inlet channel, said distal inlet channel, or said outlet channel to control pressure and/or temperature conditions at the anatomical site.

16. A method of removing materials from an anatomical site, said method comprising:
applying suction to the anatomical site via an outlet channel defined through a first sheath;
irrigating the anatomical site via a distal inlet channel defined through a tubular elongate member extendable through the first sheath; and
irrigating the anatomical site via a proximal inlet channel, defined through a second sheath through which the first sheath and the tubular elongate member are extendable.

17. The method of claim 16, further comprising shifting at least one of the first sheath, the tubular elongate member, or the second sheath with respect to the others of the first sheath, the tubular elongate member, and the second sheath.

18. The method of claim 16, further comprising fragmenting the materials with a fragmenting device.

19. The method of claim 16, further comprising withdrawing the tubular elongate member proximally within the first sheath while applying suction through the outlet channel to virtually basket materials out of the anatomical site.

20. The method of claim 16, further comprising biasing the tubular elongate member against an inner surface of the first sheath to maximize a dimension of the outlet channel transverse to a direction of fluid flow.
